(19) United States
(12) Patent Application Publication
(10) Pub. No.: US 2011/0106023 A1
(43) Pub. Date: May 5, 2011

(54) SYSTEM FOR PROVIDING TREATMENT TO A MAMMAL

(76) Inventor: Mark H. Lowe, Danville, CA (US)

(21) Appl. No.: 12/939,986

(22) Filed: Nov. 4, 2010

Related U.S. Application Data
(60) Provisional application No. 61/258,116, filed on Nov. 4, 2009.

(51) Int. Cl. A61F 7/08; A61F 7/10; A61M 35/00

(52) U.S. Cl. 604/291; 607/104

(57) ABSTRACT

There are provided thermal treatment system that utilizes a rotating coupling between a heating or chilling unit that provides a heat transfer fluid and a heat exchange bladder used to provide therapy to a mammal. The use of the rotating coupling permits the mammal to move about in a stall, assume a sleeping position or exercise while still receiving therapy.

### CROSS REFERENCE TO RELATED APPLICATIONS

This application claims priority to U.S. Provisional Application No. 61/258,116 filed on Nov. 4, 2009, entitled, “CHILLER SYSTEM FOR PROVIDING TREATMENT TO A MAMMAL.”

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

This invention generally relates to thermal treatment systems for use with mammals, especially horses.

### BACKGROUND OF THE INVENTION

Mammals are prone to diseases that require cooling and pressure techniques for treatment. One such disease affecting mammals such as horses is laminitis. Laminitis is a potentially crippling disease that has numerous precipitating causes. In the worst cases the bones of the foot penetrate the sole of the hoof which can leave the horse with significant chronic pain. 9,000 horses are euthanized annually due to the severity of the pain.

Current methods of providing cyrotherapy are ineffective either due to insufficient cooling capacity, or lack of mobility. Additionally, the extended use of the device makes it preferable to keep a cooling unit outside of the stable to prevent both the cooling unit and horse from harm. This requires extra long hoses be used to pump fluid from the cooling unit to and back from the mammal. The length of hose is bulky and awkward to store between uses.

Localized cold therapy is routinely used in the treatment of injuries such as bruises, muscle strains, sprains and similar muscle, ligament and joint dysfunctions in humans particularly for injuries to feet, ankles, legs, arms or shoulders, and in the treatment of the legs and backs of animals such as horses, particularly the lower legs.

The conventional methods of applying such localized treatment to the body portions of animals, include immersing the body portion in an ice bucket or cold water bath or the application of cold wet cloths, ice bags, or more recently chemical ice packs. Such methods are incapable of providing a sustained treatment over a relatively long period of time and present numerous other disadvantages. It should be understood that although reference is often made to animals throughout this specification, such reference should be considered to include human beings.

Despite these advances in the use of thermal therapies for the treatment of animals, there is still a need for a thermal therapy apparatus which can be used to treat multiple body parts of an animal at the same time or multiple animals at the same time while permitting the animal(s) to remain mobile or exercise while still receiving thermal therapy.

### BRIEF DESCRIPTION OF THE DRAWINGS

FIG. 1 illustrates an embodiment of a thermal therapy system using having an overhead mounted rotating coupling between the heating or cooling unit and the heat exchange bladders or appliances used to treat the front and rear legs or hooves of a horse;

FIG. 2 illustrates an embodiment of a thermal therapy system using having a counterweight biased overhead mounted rotating coupling between the heating or cooling unit and the heat exchange bladders or appliances used to treat the front and rear legs or hooves of a horse;

FIG. 3 is a perspective view of a rotating coupling having a rotating side connection to a conduit used to provide supply and return conduits to a number of heat exchange bladders or appliances;

FIG. 4 is a perspective view of various connections to a rotating coupling along with a biasing element attached to a support arm configured to move along with the rotating side of the rotating coupling;

FIG. 5 illustrates a variety of different commercially available rotating couplings that provide fluid and/or electrical connections;

FIG. 6 is a section view showing the seals and internal fluid conduits of an exemplary rotating coupling;

FIG. 7 is an isometric view of a commercially available rotating coupling showing both fluid and electrical connections;

FIG. 8 is a perspective view of a large flow covering for a chiller, as in FIG. 9;

FIG. 9 is a perspective view of a four wheeled mobile chiller unit or liquid control station;

FIG. 10 is a perspective view of a two wheeled mobile chiller unit or liquid control station;

FIG. 11 is a schematic diagram of a cold therapy apparatus according to one embodiment of the present invention;

FIG. 12 shows a detailed flat layout view of the sealed envelope of a heat exchange bladder or appliance sized and shaped for a horse's leg;

FIG. 13 shows a sectional view of the heat exchange bladder or appliance taken along lines 3-3 of FIG. 12;

FIG. 14 is another sectional view of the heat exchange bladder or appliance taken along lines 4-4 of FIG. 12;

FIG. 15 shows a detailed flat layout view of the sealed envelope of a heat exchange bladder or appliance for a horse's front leg;

FIG. 16 shows a detailed flat layout view of the sealed envelope of a heat exchange bladder or appliance for a horse's back;

FIG. 17 is a perspective view of a configured heat exchange bladder or appliance for a horse's rear leg;

FIG. 18 is a partial section view of a heat exchange bladder or appliance having both a fluid bladder and an air bladder;

FIGS. 19 and 20 are views of a heat exchange bladder or appliance having on one side a fluid bladder (FIG. 19) and on the other side an air bladder (FIG. 20);

FIG. 21 is a section view of a conduit used to provide heat exchange fluid supply and return and air supply and return;

FIG. 22 illustrates an embodiment of a thermal therapy system using having a spring biased pivoting overhead rotating coupling between the heating or cooling unit and the heat exchange bladders or appliances used to treat the front and rear legs or hooves of a horse;

FIG. 23 illustrates an embodiment of a thermal therapy system using having a ram biased pivoting overhead rotating coupling between the heating or cooling unit and the heat exchange bladders or appliances used to treat the front and rear legs or hooves of a horse;

FIG. 24 is a perspective view of a rotating coupling of FIG. 4 that includes a fluid delivery bag supported along with the rotating coupling on the opposite side of the recoil mechanism and configured to provide the fluid to the mammal receiving therapy;

FIG. 25 is a perspective view of a rotating coupling of FIG. 24 that includes a fluid delivery bag supported along with the rotating coupling on the same side as the recoil mechanism and configured to provide the fluid to the mammal receiving therapy;

FIG. 26 is a perspective view of a rotating coupling of FIG. 24 that includes a pump and drug reservoir powered and controlled via the rotating coupling and supported on the same side as the recoil mechanism and configured to pump a fluid to the mammal receiving therapy;

FIG. 27 is a plan view of a structure showing multiple stalls connected to a central liquid control station by piping for treating multiple animals at one time, including several stalls having rotating coupling type therapy connections;

FIG. 28 is a perspective view of a rotating coupling mounted on an upright to provide overhead connections to horses exercising in an arena while receiving therapy;

FIG. 29 is a perspective view of a rotating coupling mounted on an upright to provide chest height connections to horses exercising in an arena while receiving therapy;

FIG. 30 is a schematic diagram of the various conduits used to connecting a heat exchange bladder, appliance or wrap via a manifold and rotating coupling to a heating or cooling unit;

FIG. 31 is a perspective view of a manifold connected to an overhead mounted rotating coupling;

FIG. 32 is a perspective view of a manifold connected to an overhead mounted rotating coupling;

FIG. 33 is a perspective view of a manifold connected to an overhead mounted rotating coupling with pivoting connections to four heat exchange bladders or appliances;

FIG. 34 is a perspective view of a manifold connected to an overhead mounted rotating coupling having a pivoting or rotating connection on the manifold;

FIG. 35 is a perspective view of a manifold connected to an overhead mounted rotating coupling with a manifold connector angled towards the orientation of the rotating coupling;

FIG. 36 is a perspective view of a manifold having a connection directed towards an overhead mounted rotating coupling;

FIG. 37 is a perspective view of a manifold shaped for placement on a mammal having an insulated layer and a moveable top mounted connection directed towards an overhead mounted rotating coupling;

FIG. 38 illustrates an embodiment of a thermal therapy system using having an overhead mounted rotating coupling connected to a back mounted manifold that is connected to four heat exchange bladders or appliances used to treat the front and rear legs or hooves of a horse;

FIG. 39 illustrates an embodiment of a thermal therapy system using having a chest level mounted rotating coupling connected to a chest mounted manifold that is connected to four heat exchange bladders or appliances used to treat the front and rear legs or hooves of a horse.

### SUMMARY OF THE INVENTION

The present invention relates to a cooling system for providing treatment to a mammal on the move. One aspect of the invention comprises a long hose (possibly greater than 20 feet) and a multi-port swivel joint for full range of motion of the mammal without cross tying. Without such a swivel, the horse will eventually wind up the hose and either kink or pull the hose loose once the slack is eliminated. The swivel could include a moment arm to reduce torque required to rotate the swivel joint. The swivel joint could include electrical connections to pass electrical signals from the horse to the chiller for easy remote monitoring of the horse.

Another aspect of the invention comprises a method of storing extra hose for more range of motion of a mammal (i.e. if the mammal wanted to lie down.) The method may include a counterbalance weight attached to a chilling unit and spring loaded retractable cord attached to moment arm of swivel.

Another aspect of the invention includes drip-less couplings to minimize spillage of the glycol solution. Flavor may be added to chiller coolant to discourage animals from drinking spilled fluid. In addition, extra venting may be added in side panels for higher efficiency cooling.

Another aspect of the invention comprises large wheels (possibly greater than 6 inches) to be maneuverable over uneven terrain. Versatile handles can be used to push or pull the product, or to lift the product up over high thresholds, or curbs. Handles may serve as a hanging rack for long hoses. The hose may be up to 30 ft.

In one aspect, there is provides a treatment system for a mammal having a rotating coupling having a fixed side supply conduit, a fixed side return conduit, a rotating side supply conduit, and a rotating side return conduit. There is a heat exchange bladder for the mammal being treated by the thermal treatment system, the heat exchange bladder having a supply conduit and a return conduit. There is also a heating or cooling unit having a heat exchange fluid supply conduit and a heat exchange fluid return conduit. A hose connects the heat exchange bladder supply conduit to the rotating side supply conduit. A hose connects the heat exchange bladder return conduit to the rotating side return conduit. A hose connects the fixed side supply conduit to the heat exchange fluid supply conduit and a hose connects the fixed side return conduit to the heat exchange fluid return conduit. There may also be a biasing element connected to the rotating coupling or at least one of the hose connecting the heat exchange bladder supply conduit to the rotating side supply conduit and the hose connecting the heat exchange bladder return conduit to the rotating side return conduit. In one alternative, at least a portion of the biasing element moves along with the movement of the mammal being treated by the treatment system. In one aspect, the biasing element is connected to the rotating coupling. In various alternatives, the biasing element is one of a counterweight system, a spring loaded return coil, a spring, a hydraulic ram and a pneumatic ram. In one alternative, the biasing element is configured to maintain a first position when the mammal is receiving therapy in a standing position and a second position when the mammal is receiving therapy in a non-standing position.

In one aspect there is also an arm connected to the rotating coupling and the biasing element supported by the arm. In another aspect, there is also a pivoting connection on the arm wherein the biasing element is connected to the aim to control the movement of the arm about the pivoting connection.

In still other embodiments, the therapy system includes an upright support connected to the rotating coupling and another heat exchange bladder for another mammal. In addition, the rotating coupling includes another rotating side supply conduit, and another rotating side return conduit wherein the another rotating side supply conduit and the another rotating side return conduit are connected to the another heat exchange bladder. In one embodiment, a hose connecting the fixed side supply conduit to the heat exchange fluid supply conduit and a hose connecting the fixed side return conduit to the heat exchange fluid return conduit are within or along the upright support. In addition, the upright support has a height that places the rotating coupling near the chest level of the mammal receiving therapy from the treatment system. Alternatively, the upright support has a height that places the rotating coupling above the head of the mammal receiving therapy from the treatment system. In still other aspects, the upright support and the rotating coupling are positioned to permit the mammal and the another mammal to move in a generally circular path about the upright support.

In still other embodiments, the treatment system includes an air bladder on the heat exchange bladder; an air supply port on the rotating coupling; and an air supply line between the air bladder and the air supply port. In another alternative, there is a pharmacological agent in a container in fluid communication with rotating coupling to deliver the pharmacological agent to an outlet on the rotating side of the rotating coupling and a delivery conduit from the outlet on the rotating side to the mammal. In one aspect, there a container mounted to the rotating side of the rotating coupling, the container including a pharmacological agent and having a delivery conduit in communication with the mammal receiving therapy. In still other embodiments, there is an electrical connection providing through the rotating coupling to a component that provides, for example, power to the component or a signal path for the component. The component may be a pump, a sensor on the mammal, such as one that provides an indication of the mammal receiving therapy. In some embodiments, the sensor is one of an ECG lead, a respiration sensor, a blood pressure sensor or a temperature sensor. In one aspect, the component is a sensor measuring an indication of the therapy system. In still another aspect, the hose connecting the heat exchange bladder supply conduit to the rotating side supply conduit or the hose connecting the heat exchange bladder return conduit to the rotating side return conduit has a rotational stiffness of between about 1 in-lb/rad to about 25 in-lb/rad.

In one embodiment of the invention there is provided a treatment system for a mammal having a rotating coupling having a fixed side supply conduit, a fixed side return conduit, a rotating side supply conduit, and a rotating side return conduit. There is at least one heat exchange bladder for the mammal being treated by the thermal treatment system, the heat exchange bladder having a supply conduit and a return conduit. There is also a manifold having a main supply conduit, a main return conduit, at least one auxiliary supply conduit and at least one auxiliary return conduit. There is also a heating or chilling unit having a heat exchange fluid supply conduit and a heat exchange fluid return conduit. Several hoses are provided to complete the fluid circuit such as, for example, a hose connecting the manifold main supply conduit to the rotating side supply conduit; a hose connecting the manifold main return conduit to the rotating side return conduit; a hose connecting the at least one auxiliary supply conduit to the heat exchange bladder supply conduit; a hose connecting the at least one auxiliary return conduit to the heat exchange bladder return conduit; a hose connecting the fixed side supply conduit to the heat exchange fluid supply conduit; and a hose connecting the fixed side return conduit to the heat exchange fluid return conduit.

In still another aspect, the treatment system for a mammal is for a horse and there is a surface on the manifold configured to conform to a portion of the back of the horse and the length of the hose connecting the at least one auxiliary supply conduit to the heat exchange bladder supply conduit and the length of the hose connecting the at least one auxiliary return conduit to the heat exchange bladder return conduit are selected to extend from the manifold to a hoof of the horse. In another aspect, in use, the hose connecting the manifold main supply conduit to the rotating side supply conduit or the hose connecting the manifold main return conduit to the rotating side return conduit extend from the manifold in a direction towards the rotating coupling.

In another alternative, the treatment system for a mammal is for a horse and a surface on the manifold configured to conform to a portion of the chest of the horse and the length of the hose connecting the at least one auxiliary supply conduit to the heat exchange bladder supply conduit and the length of the hose connecting the at least one auxiliary return conduit to the heat exchange bladder return conduit are selected to extend from the manifold to a hoof of the horse.

In still another alternative, the treatment system includes a biasing element connected to the rotating coupling or to at least one of the hose connecting the manifold main supply conduit to the rotating side supply conduit and the hose connecting the manifold main return conduit to the rotating side return conduit. In one alternative, the biasing element moves along with the movement of the mammal being treated by the treatment system. In another alternative, the biasing element is connected to the rotating coupling. In one aspect, the biasing element is one of a counterweight system, a spring loaded return coil, a spring, a hydraulic ram and a pneumatic ram. In one alternative, there is an arm connected to the rotating coupling and the biasing element supported by the arm. In still another alternative, there is a pivoting connection on the arm wherein the biasing element is connected to the arm to control the movement of the arm about the pivoting connection. In one aspect, the biasing element is configured to maintain a first position when the mammal is receiving therapy in a standing position and a second position when the mammal is receiving therapy in a non-standing position.

In one aspect, the treatment system also includes an upright support connected to the rotating coupling; another heat exchange bladder for another mammal. The rotating coupling also has another rotating side supply conduit, and another rotating side return conduit wherein the another rotating side supply conduit and the another rotating side return conduit are connected to the another heat exchange bladder. Still further, the hose connecting the fixed side supply conduit to the heat exchange fluid supply conduit and the hose connecting the fixed side return conduit to the heat exchange fluid return conduit are within or along the upright support. In another aspect, the upright support has a height that places the rotating coupling near the chest level of the mammal receiving therapy from the treatment system. In another aspect, the upright support has a height that places the rotating coupling above the head of the mammal receiving therapy from the treatment system. In another aspect, the upright support and the rotating coupling are positioned to permit the mammal and the another mammal to move in a generally circular path about the upright support. In still another alternative, the hose connecting the manifold main supply conduit to the rotating side supply conduit or the hose connecting the manifold main return conduit to the rotating side return conduit has a rotational stiffness of between about 1 in-lb/rad to about 25 in-lb/rad.

In still other additional embodiments, there is a method of providing therapy to a mammal by attaching a heat exchange bladder to a portion of the mammal. Next, placing the heat exchange bladder in fluid communication with a rotating coupling and the rotating coupling in fluid communication with a heating or chilling unit. Thereafter, circulating heat exchanging fluid from the heat exchanger through the rotating coupling to the heat exchange bladder while movement performed by the mammal rotates the rotating coupling. In one alternative, the method includes displacing the rotating coupling as the mammal moves from a first position to a second position. In one aspect, the first position or the second position the mammal is in a sleeping position. In another aspect, the method includes extending a biasing element during the displacing step when the rotating coupling is displaced about a pivot point connected to the rotating coupling. In other aspects, during the circulating step the rotating coupling is maintained above the head of the mammal receiving therapy or at about chest level on the mammal. In still another aspect, the method includes performing the attaching step, the placing step and the circulating step with another mammal such that circulating step is performed using one rotating coupling. In another aspect, the method includes moving the mammal and the another mammal in a generally circular path about the rotating coupling during the circulating step. In one aspect, the displacing step includes displacing the rotating coupling about a pivot point.

### DETAILED DESCRIPTION OF THE INVENTION

The subject matter of the present application is related to subject matter described in: U.S. patent application Ser. No. 09/127,256 (filed Jul. 31, 1998) entitled, “Compliant Heat Exchange Panel” issued on Apr. 3, 2007 as U.S. Pat. No. 7,198,093; U.S. patent application Ser. No. 09/798,261 (filed Mar. 1, 2001) entitled, “Shoulder Conformal Therapy Component of an Animate Body Heat Exchanger”; U.S. patent application Ser. No. 09/901,963 (filed Jul. 10, 2001) entitled, “Compliant Heat Exchange Splint and Control Unit”; U.S. patent application Ser. No. 09/771,123 (filed Jan. 26, 2001) entitled, “Wrist/Hand Conformal Therapy Component of an Animate Body Heat Exchanger”; U.S. patent application Ser. No. 09/771,124 (filed Jan. 26, 2001) entitled, “Foot/Ankle Conformal Therapy Component of an Animate Body Heat Exchanger”; U.S. patent application Ser. No. 09/771,125 (filed Jan. 26, 2001) entitled, “Conformal Therapy Component of an Animate Body Heat Exchanger having Adjustable Length Tongue”; U.S. patent application Ser. No. 10/784,489 (filed Feb. 23, 2004) entitled, “Therapy Component of an Animate Body Heat Exchanger” which is a continuation of U.S. patent application Ser. No. 09/765,082 (filed Jan. 16, 2001) entitled, “Therapy Component of an Animate Body Heat Exchanger and Method of Manufacturing such a Component” issued on Feb. 24, 2004 as U.S. Pat. No. 6,695,872 which is a continuation-in-part of U.S. patent application Ser. No. 09/493,746 (filed Jan. 28, 2000) entitled, “Cap And Vest Garment Components Of An Animate Body Heat Exchanger” issued on Jan. 30, 2001 as U.S. Pat. No. 6,178,562; U.S. patent application Ser. No. 10/122,469 (filed Apr. 12, 2002) entitled, “Make-Break Connector For Heat Exchanger” issued on Mar. 29, 2005 as U.S. Pat. No. 6,871,878; U.S. patent application Ser. No. 10/637,719 (filed Aug. 8, 2003) entitled, “Apparel Including a Heat Exchanger” issued on Sep. 19, 2006 as U.S. Pat. No. 7,107,629; U.S. patent application Ser. No. 12/208,240 (filed Sep. 10, 2008) entitled, “Modular Apparatus for Therapy of an Animate Body” which is a divisional of U.S. patent application Ser. No. 10/848,097 (filed May 17, 2004) entitled, “Modular Apparatus for Therapy of an Animate Body”; U.S. patent application Ser. No. 11/707,419 (filed Feb. 13, 2007) entitled, “Flexible Joint Wrap”; U.S. patent application Ser. No. 11/854,352 (filed Sep. 12, 2007) entitled, “Make-Break Connector Assembly with Opposing Latches”, U.S. patent application Ser. No. 12/329,461 (filed Dec. 5, 2008) entitled, “Cooling System Having A Bypass Valve To Regulate Fluid Flow”; U.S. patent application Ser. No. 12/329,481 (filed Dec. 5, 2008) now U.S. Patent Application publication 2010/0145421, entitled, “Therapeutic Cooling And/Or Heating System Including A Thermo-Conductive Material” which is incorporated herein by reference.

### System for Providing Treatment to a Mammal

While aspects of the specification relate to embodiments where the heating or cooling unit is a chiller, it is to be appreciated that embodiments of the invention are not so limited. The embodiments of the present invention may find applicability in various forms of thermal therapy whether for a heat based therapy or a cold based therapy. For purposes of illustration, the specification will describe a heating or cooling unit that is configured as a chiller. The chiller system provides a clinically effective cooling medium or heat transfer fluid to the impacted hoof or feet of the mammal during a cold therapy application, such as a laminitis treatment regime for a horse. Embodiments of the present invention allow mobility and maneuverability in a variety of environments.

One aspect of the invention provides cyrotherapy and cooling techniques while providing mobility of a mammal especially during light or unattended use. In embodiments and examples below, a horse will be used as an exemplary mammal. However, any type of mammal may be substituted for the horse.

Another aspect of the invention provides a swivel joint or rotating coupling that can accommodate more than one fluid line in order to allow free access to the horse for extended treatment with the chilling system. The rotating coupling has a fixed side and a rotating side. The sides each contain one or more fluid connections and/or electrical connections. In use, a fluid or electrical connection made on the fixed side is maintained to a designated connection on the rotating side. The connection remains while the rotating side and its respective electrical or fluid connections rotate relative to the fixed side. The rotating coupling is used to provide fluid and/or electrical connectivity between the heating or cooling unit and the appliance or heat exchange bladder that is delivering therapy to the mammal.

The rotating coupling has a fixed side supply conduit, a fixed side return conduit, a rotating side supply conduit and a rotating side return conduit. In addition, the rotating coupling may also provide an electrical connection through the coupling to a component. As used herein, an electrical connection refers to the ability of the rotating coupling to provide electrical power or provide a signal pathway or pass analog or digital signals between the fixed and rotating sides of the coupling. The fixed side may be further connected to a system controller or other feedback or control system. The rotating side may be connected to a component. The component may be a pump such as an infusion pump to deliver a pharmacological agent to the mammal. The pharmacological agent can be supplied from fixed side, through the rotating coupling, or attached to the rotating side as desired. The pharmacological agent may be any A biologically active substance applied pharmacologically to the mammal for their therapeutic effects on one or more tissues or organs as an independent action or in conjunction with the thermal therapy provided by the system. The pharmacological agent may provide an anti-inflammatory response or be an anti-bacterial agent. Alternatively, the pump or the therapy system could be configured to provide one or more fluids to the mammal such as saline or other fluids to provide nutrition or hydration to the mammal.

The component may be any of a variety of temperature, pressure or electrical sensors. Examples of sensors used to monitor the mammal include ECG electrodes, blood pressure monitors, and temperature sensors. Examples of sensors used to monitor the system include temperature sensors, pressure sensors or flow sensors. The type of electrical connection depends on the configuration of the thermal therapy system. The component may be used to monitor or further treat the animal or monitor or control a portion of the thermal treatment system.

One embodiment is a hose swivel or rotating coupling with a single fluid channel. The fluid may pass through the hose swivel axis, sealing the joint with a single o-ring. Another embodiment is a hose swivel with multiple fluid channels.

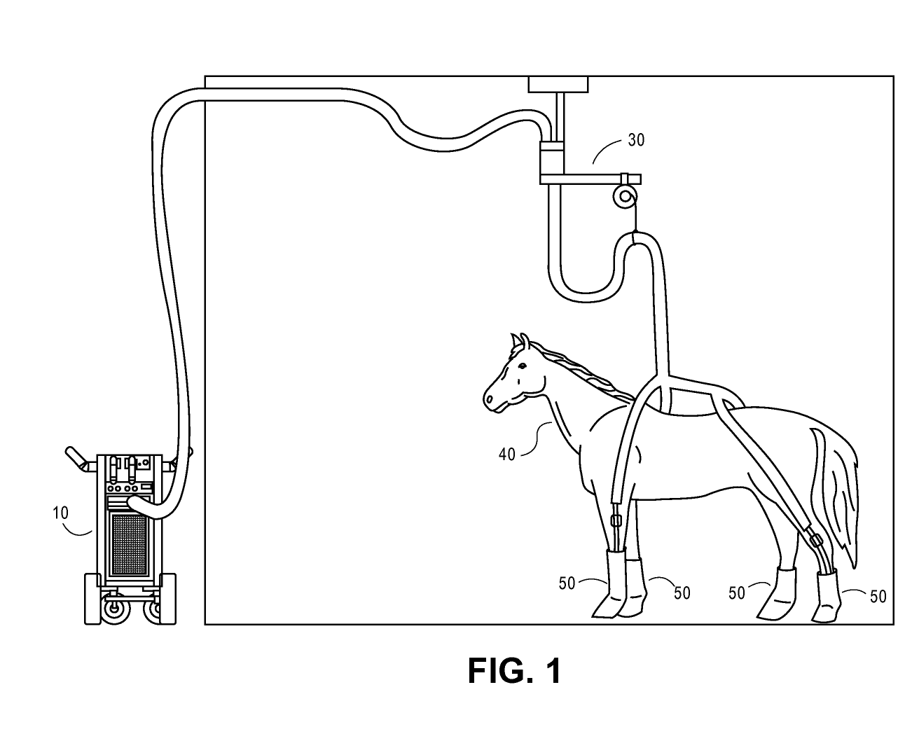

FIG. 1 illustrates an embodiment of a thermal therapy system using having an overhead mounted rotating coupling between the heating or cooling unit and the heat exchange bladders or appliances used to treat the front and rear legs or hooves of a horse. FIG. 1 illustrates a chiller unit 10, hose 20, a rotating coupling 30 and a mammal 40. The chiller unit 10 may be located in a different area than the mammal 40 (such as outside of a horse stall wall). The hose 20 is attached to the chiller unit 10 and supplies coolant to and from the wrap 50 (i.e., hoof heat exchanger). The wrap may be similar to in the above references incorporated by references above. The addition of another fluid channel for pneumatic pressure is also desirable to add compression to the wrap 50 allowing for better contact between a mammal contact area and wrap 50.

The rotating coupling 30 may be replaced by a coupling that provides any type of movement.

A first section of hose 20 is connected to the chiller unit 10 and may be routed up over a wall (i.e., horse stall wall), along the ceiling. The hose 20 is then connected to a rotating coupling 30. The end of the rotating coupling 30 most proximal to the horse is again attached to a second section of hose 20 that connects to the wrap 50. The second section of hose 20 may connect to one or more wraps as desired.

In FIG. 1, the horse has four wraps and the hose is split into four ends to accommodate the four wraps.

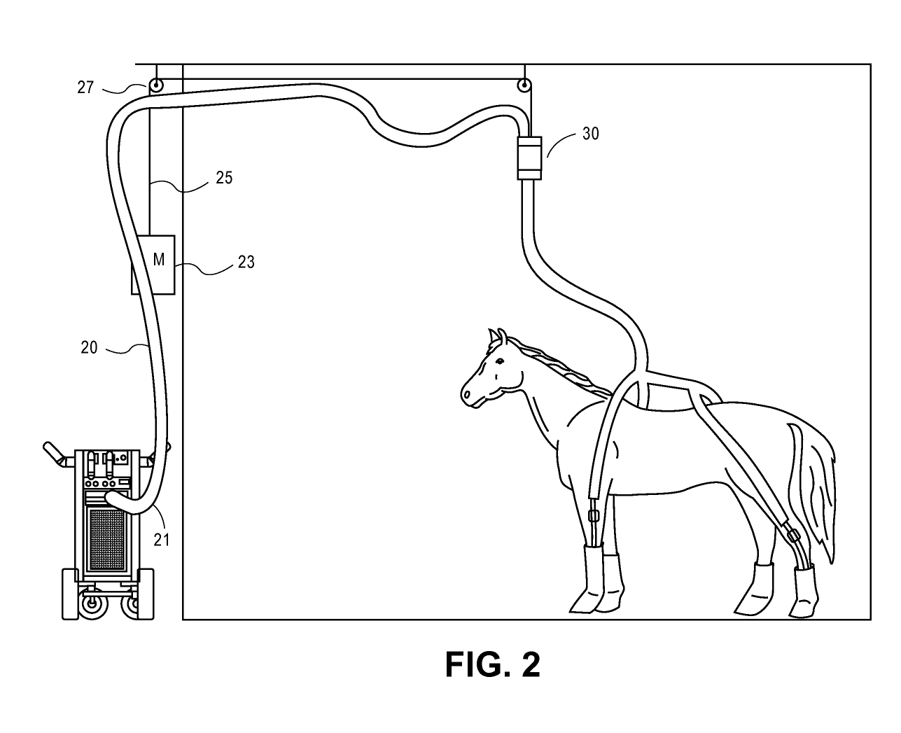

FIG. 2 illustrates an embodiment of a thermal therapy system using having a counterweight biased overhead mounted rotating coupling between the heating or cooling unit and the heat exchange bladders or appliances used to treat the front and rear legs or hooves of a horse.

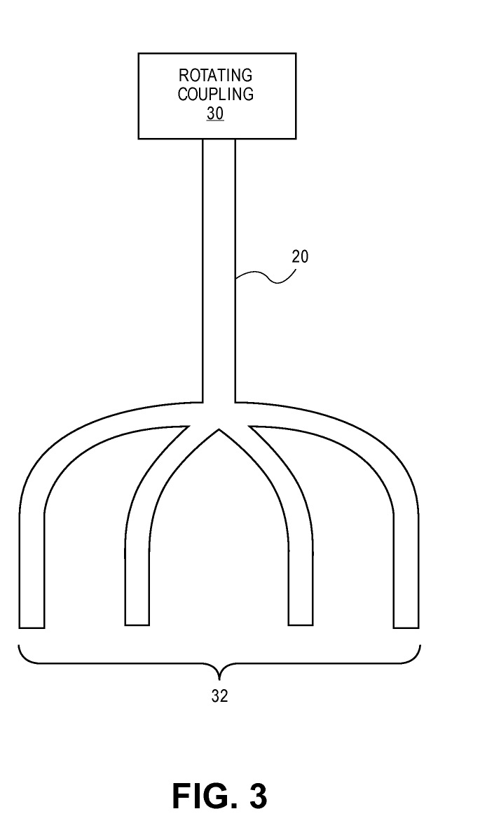

FIG. 3 is a perspective view of a rotating coupling having a rotating side connection to a conduit used to provide supply and return conduits to a number of heat exchange bladders or appliances. FIG. 3 illustrates that the hose 20 may be split into various sections 32 for delivery of coolant and/or compression into a wrap 50. Any combination of hose section(s) 32 may be combined with any combination of wrap(s) 50.

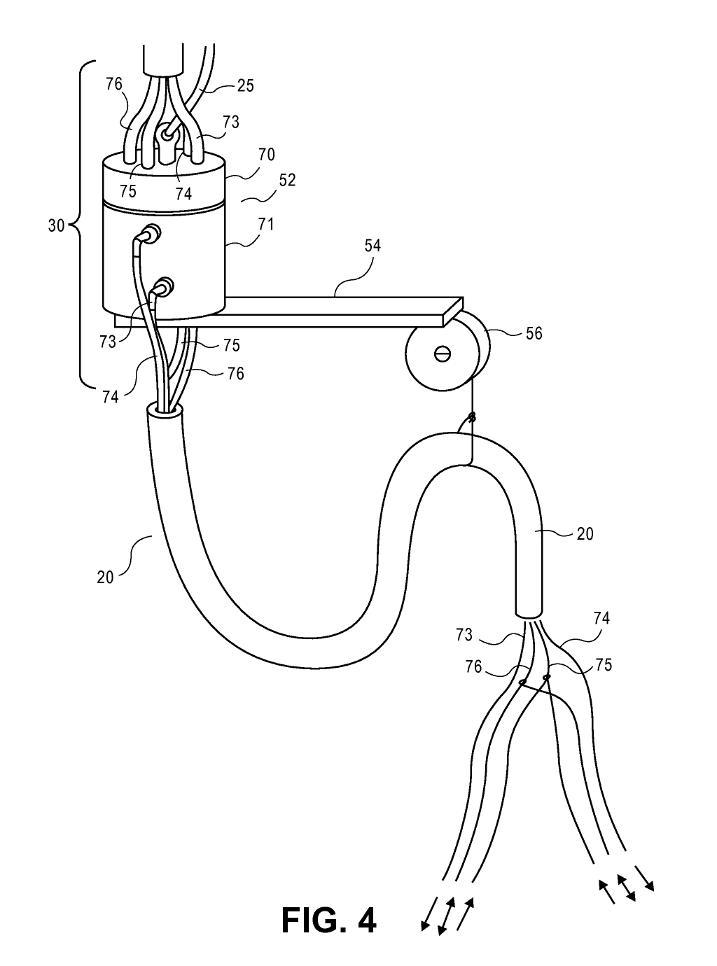

FIG. 4 is a perspective view of various connections to a rotating coupling along with a biasing element attached to a support arm configured to move along with the rotating side of the rotating coupling. FIG. 4 shows a detailed view of the rotating coupling 30 including a rotary union 52, moment arm 54, and cord retractor 56. In FIGS. 1 and 4, an optional moment arm 54 is attached to the second end of the rotating coupling 52, allowing for easier swiveling. This embodiment may be used if the friction of the union is more than the mammal can apply when turning in circles.

FIG. 4 also shows a fluid arrangement with two fluid supply lines 73 and 74, one fluid return line 75 and one bidirectional air line 77. The fluid lines connect to a fixed portion 70 of rotary union 52 and exit out a rotationally free portion 71 of rotary union 52. Furthermore, the fluid lines may be split into two or more fluid paths. FIG. 4 shows the hose 20 being split into two hoses to treat 2 hooves. This could also be further split again to serve 2 more hooves.

Yet another aspect of this invention involves a method to allow for extra slack to be delivered in case the mammal lies down. The slack may be provided by the long length of the hose 20 or by flexible material of the hose 20. Without such a method, either the hose would be pulled down from the ceiling, or there would have to be too much slack in the hose when the horse was in the normal standing position. The extra slack in the hose would be hazardous, could be easily chewed by the horse, or at least would be in the way.

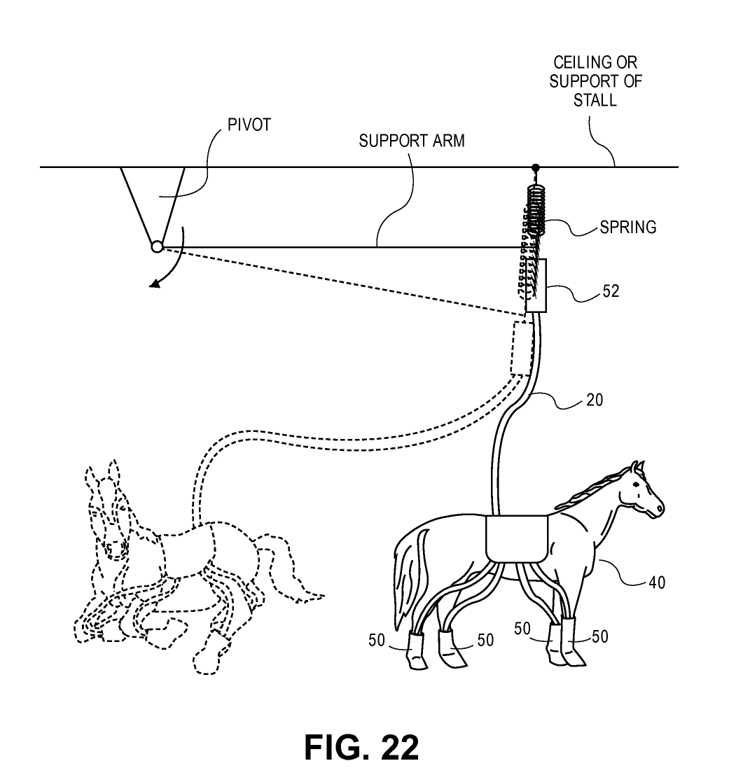

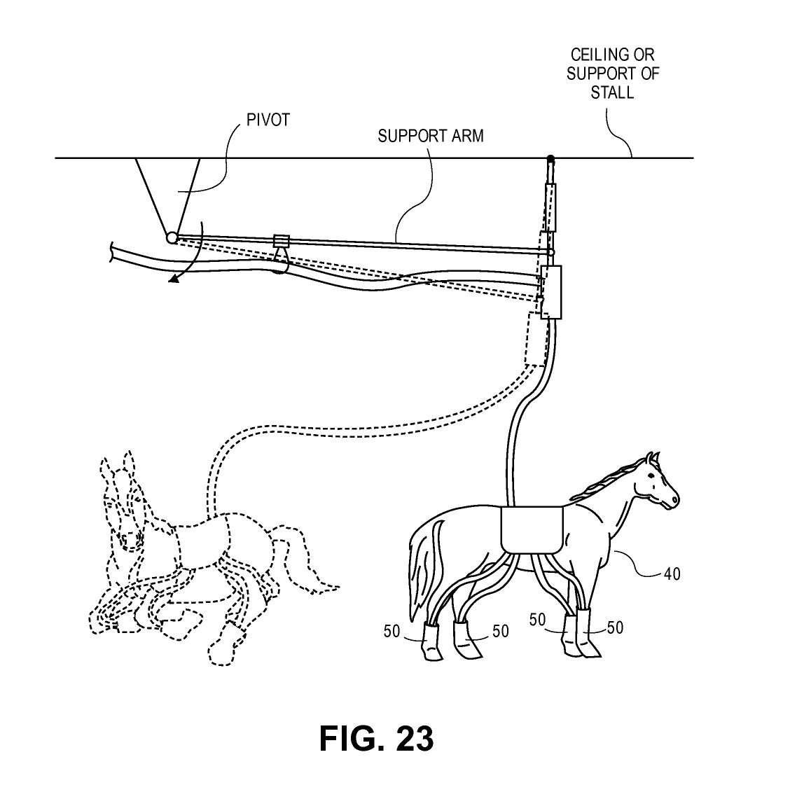

FIGS. 1 and 4 also show a cord retractor 56 mounted to the rotating coupling 30. This allows the mammal to lie down without damaging or breaking the coolant hose 20 or rotary union 52. Alternatively, the cord retractor 56 can be mounted to the ceiling and attached to the rotary union 52. The cord retractor 56 could also be a bungee cord, or a long flexible pole oriented horizontally, or an arm with a pivot (see FIGS. 22 and 23).

FIG. 2 shows yet another method of removing slack. Rotating coupling 30 is attached to a support line 25 and suspended by one or more pulleys 27. The weight of the hose is balanced by counterweight 23. If the horse lies down, the support line 25 is pulled and the counterweight rises, and extra slack 21 in hose 20 is released.

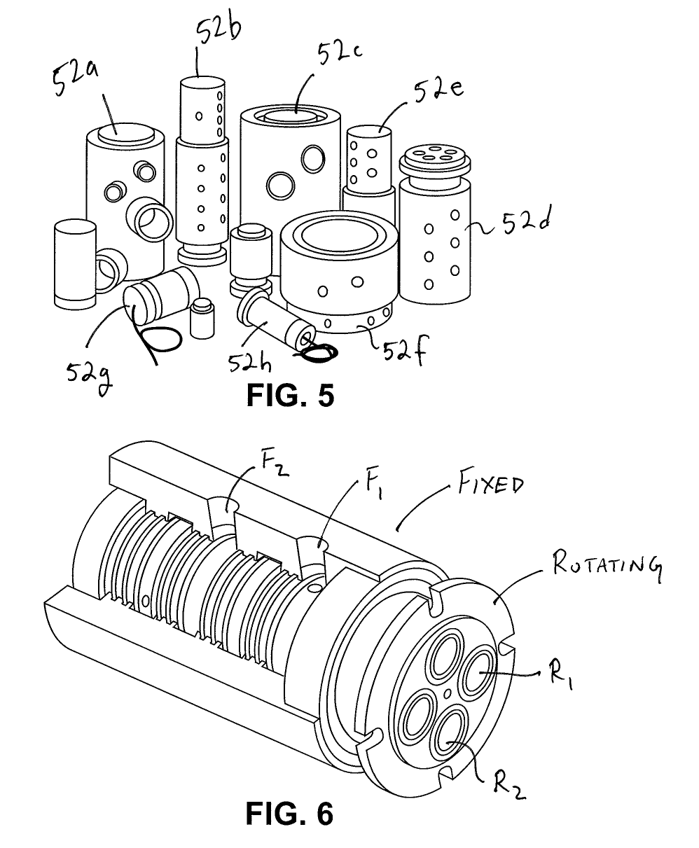

FIG. 5 illustrates a variety of different commercially available rotating couplings that provide fluid and/or electrical connections. FIG. 5 shows examples of rotating couplings or rotary unions 52a-52h. Rotating couplings 52a-52f illustrate a variety of different fluid rotating couplings of various sizes and number and orientation of ports or conduits. Couplings 52g and 52h illustrate various configurations for electrical connectivity via a rotating coupling. Rotating couplings, also referred to as rotating unions, are available commercially from Dynamic Sealing Technologies, Inc. of Andover, Minn.

FIG. 6 is a section view showing the seals and internal fluid conduits of an exemplary rotating coupling. FIG. 6 shows a cutaway view of an exemplary rotating coupling or multiport rotary union 52 in a cutaway view. The coupling 52 has a fixed side (F) and a rotating side (R). Has shown, the fixed side fluid ports F1 and F2 are connected using conduits and seals within the coupling to the respective ports on the rotating side R1 and R2.

Another aspect of the invention utilizes electrical slip rings to return electrical signals from the mammal foot (i.e., horse hoof) back to the chiller unit 10 for easier monitoring. The electrical signals may be temperature sensor signals. It has been valuable to note the foot (or hoof) temperature during treatment to ensure that the treatment is effective. Additionally, ambient temperature and heart rate could be monitored. It is preferable to include a foot (or hoof) temperature display back to the chiller, outside the stall, or to a remote location.

Bringing an electrical signal back to the chiller unit 10 presents similar problems as with the coolant hose 20, and the electrical wires may eventually become tangled or twisted. Integrating the rotary union 52 with electrical connectors utilizing slip rings inside the rotary union 52 solves this problem. Alternatively, a wireless system could be developed using any number of available wireless standards.

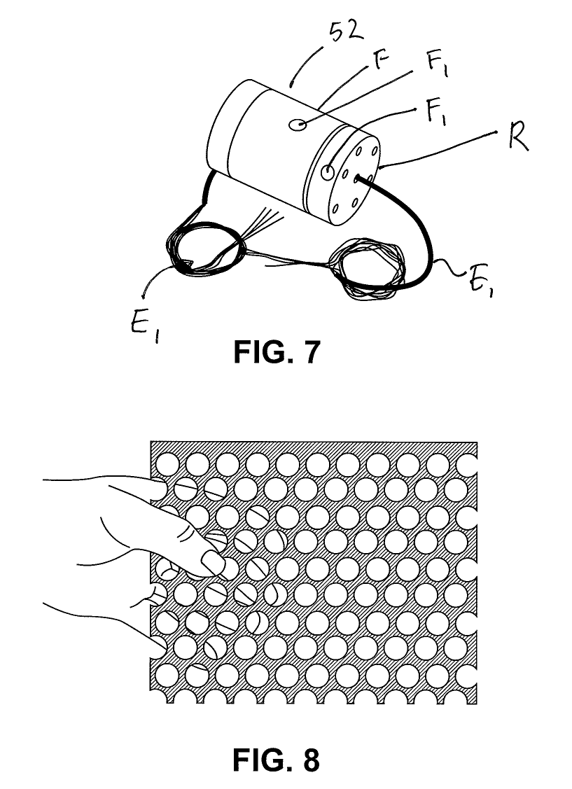

FIG. 7 is an isometric view of a commercially available rotating coupling showing both fluid and electrical connections. FIG. 7 is an isometric view of a rotating union or rotating coupling 52 with an electrical slip ring (or other suitable electrical connection suited to maintain electrical connectivity during rotation) and fluid connections. The rotating coupling 52 has a fixed side F and a rotating side R. Electrical wiring E1 on the fixed side F is in electrical communication with rotating side electrical wiring E1 on rotating side R. The fixed side fluid port F1 is in communication with rotating side fluid port F1.

### Heating or Cooling Unit

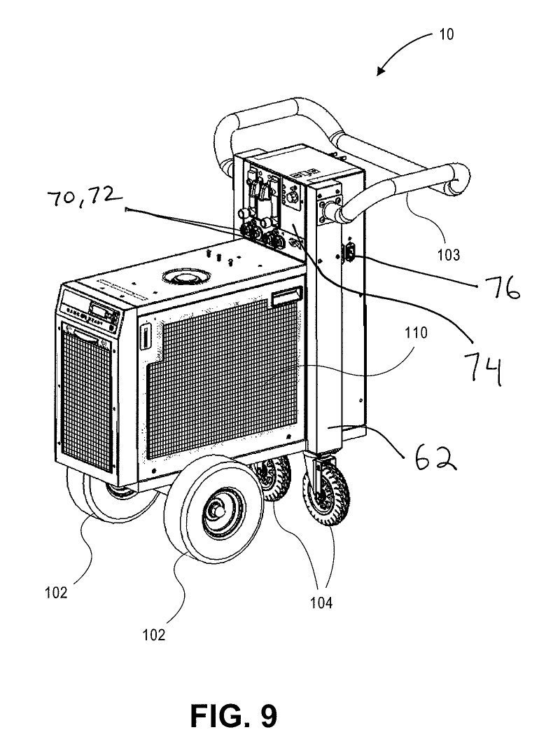

FIG. 9 is a perspective view of a four wheeled mobile chiller unit or liquid control station. FIG. 9 illustrates an exemplary chiller unit 10. The chiller unit 10 has 4 wheels, including at least 2 large diameter wheels 102, allowing for easy transportation, even over rough surfaces. The preferred embodiment utilizes two swivel castors 104 to allow for easy navigation of the system in tight quarters. Preferably the swivel castors are of the locking variety, preventing unwanted movement of the system during use. Alternatively, the chiller unit 10 may have any number of wheels or swivel castors or may have no wheels or swivel castors.

It is also preferable for the wheels and castors to be soft enough to provide cushioning effect to minimize vibration when being wheeled across bumpy terrain, or when being shipped from site to site in a pick up truck, or by common carrier.

The handle 103 provides good leverage when the system is being moved by the user. The handle 103 can be used to lift the rear castors over curbs or large thresholds, to pull the chiller over rough, gravelly surfaces, and used to push the chiller on smooth surfaces.

The handle 103 also has wide cradle shaped areas to hang or drape the long coiled hoses when not in use, or to store extra hose during use. This allows for the hoses to be kept off the ground for sanitary reasons.

In addition, the chiller unit 10 may also include front panels 109 and side panels 110 with a high density perforated hole pattern as noted above to allow for superior air flow (see e.g., the panel illustrated in FIG. 8).

In some thermal applications, high power may be required to remove the appropriate amount of heat from the mammal foot or hoof is such that most chillers cannot deliver the proper amount of cooling capacity through standard 115VAC 15A circuit. Additional electrical wiring to accommodate a larger more powerful chiller may be provided. However, a standard chiller may be modified to use a high density perforated hole pattern (or other open air flow pattern) for its side and front panels (and/or its entire enclosure), more air can flow thru the evaporator coils. Higher air flow allows for more efficient cooling. This allows a chiller to use standard line voltage and amperage, eliminating the need for special electrical wiring.

A representative high density perforated pattern can be seen in FIG. 8. Other open patterns or methods may be used, but they should be compatible with the appropriate mechanical and electrical safety standards.

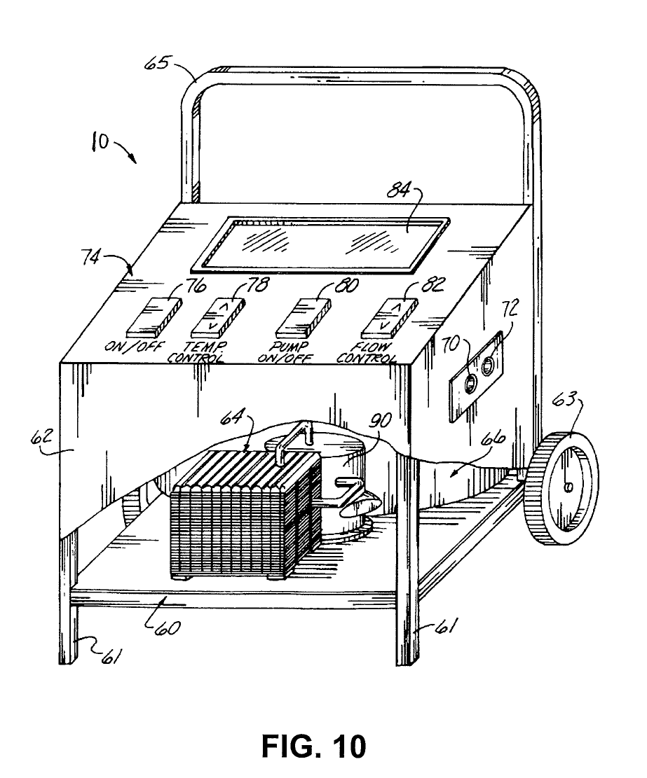

FIG. 10 is a perspective view of a two wheeled mobile chiller unit or liquid control station. The portable chiller or liquid control station (10) comprises a main frame (60) surrounded by a housing (62). Attached to the bottom of the housing are a pair of pneumatic rear wheels (63) and front legs (61). A handle (65) is mounted to the main frame (60) to allow the station (10) to be transported and positioned in close proximity to the animal to be treated by tilting the appliance so it can be wheeled much like a two-wheeled dolly cart. It should be appreciated that a pair of front wheels could replace the front legs (61) resulting in a four-wheeled cart, as described above in FIG. 9.

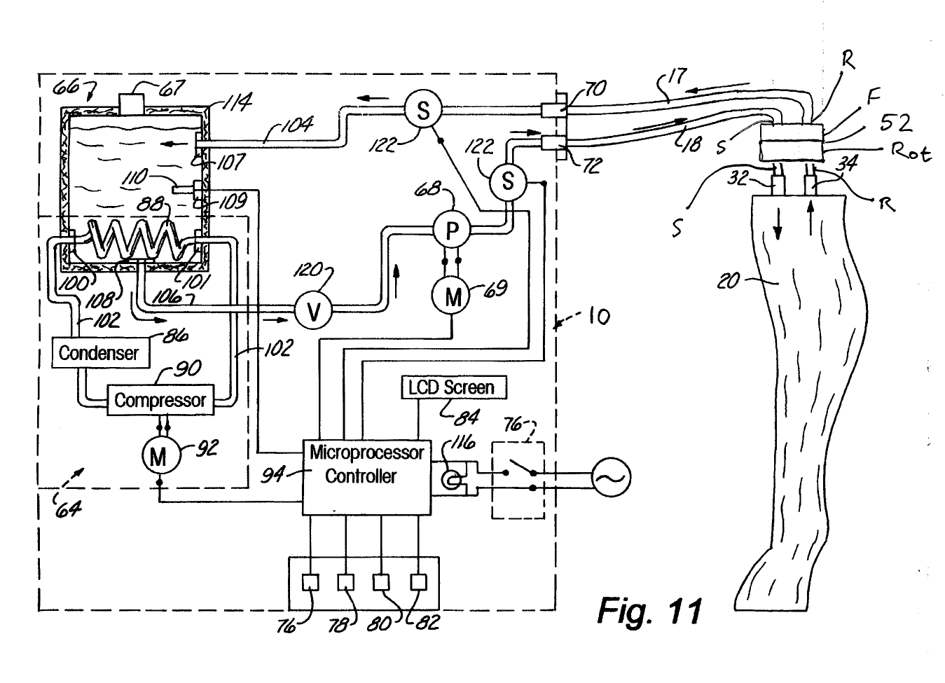

Many of the elements of the control station (10) are hidden in FIG. 8 and are schematically illustrated in FIG. 11. FIG. 11 is a schematic diagram of a cold therapy apparatus according to one embodiment of the present invention. The station (10) contains a refrigeration system (64), a liquid cooling reservoir (66), a liquid pump (68), a liquid pump motor (69), a supply port (72) and an return port (70) projecting through the side of the housing (62). A control panel (74) located on the top of the housing (62) includes a main power on/off switch (76), a temperature controller (78), a pump on/off switch (80), a flow or pressure controller (82) and a liquid crystal display (LCD) screen (84). Electrical power for operating the control station (10) is preferably supplied by an external 120 volt AC power source but electrical power could also be supplied by an internal combustion engine operating an electric generator.

The refrigeration system (64) includes a condenser (86), evaporator coils (88) disposed in the bottom of the liquid reservoir (66), a compressor (90), and a compressor motor (92).

The liquid reservoir (66) is a tank preferably having a five gallon capacity and constructed of polymeric material such as polyethylene. A sleeve (67) is provided near the top of the tank for filling the liquid reservoir with the liquid to be circulated through the system. It is preferred to use potable water in the system because it is readily available in most animal facilities and it is non-toxic in the case of spills or rupture of the heat exchange bladder or appliance (19, 20 or 21) or return and supply conduits (17 and 18). Non-toxic propylene glycol may also be mixed with the water if temperatures below freezing are desired for treatment. Liquid tight fittings (100, 101) are required for the passage of the refrigerant tubing (102) between evaporation coils (88) and compressor (90) and condenser (86). Supply and return piping (106 and 104 respectively) are coupled to the reservoir (66) also by liquid tight fittings (107 and 108) on the tank wall and bottom respectively. Additional liquid tight fittings (109) are required for the tank thermal sensors (110) (discussed below). Thermal insulation (114) in blanket or spray-on form is used to surround the liquid reservoir (66) to increase thermal efficiency.

The liquid circuit includes a supply line (106), preferably constructed out of flexible, synthetic, reinforced rubber, in communication with the liquid reservoir tank (66). A liquid pump (68) is connected to the supply line (106). The pump is operated by the pump motor (69). Intermediate the reservoir tank (66) and pump (68) is a one way check valve (120) to prevent back-flow of the cooled liquid into the reservoir (66). The supply line (106) terminates with a quick disconnect coupler at the supply port (72).

The rotating coupling 52 is connected between the chiller 10 and the heat exchange bladder or appliance 20. A fixed side supply conduit (18) is coupled at one end to the supply port (72) of the control station (10) and at its other end to a fixed side F supply port S. A fixed side return conduit 17 is coupled at one end to the return port 70 and at its other end to the fixed side F return port R. A rotating side supply conduit is connected to the rotating side Rot supply port S and the supply conduit or inlet 32 of the heat exchange bladder or appliance (19, 20 or 21). The heat exchange bladder or appliance return conduit 34 is connected to the rotating side Rot return port R. All connections may be quick disconnect style coupler connections. A return line (104), preferably constructed out of flexible synthetic reinforced rubber, in communication with the reservoir tank (66) and return port (70) returns the used water from the heat exchange bladder or appliance (19, 20 or 21) to the reservoir tank (66).

The temperature controller (78) is used to set the desired temperature for the liquid in the reservoir (66). A thermal sensor (110) is disposed in the reservoir (66) to monitor the temperature of the liquid. The thermal sensor (110) is electrically coupled to a microprocessor controller (94) which controls the compressor motor (92). The temperature controller (78) and LCD screen (84) are likewise coupled to the microprocessor (94) so the user can view the temperature settings and actual temperatures. The necessary mechanical and electrical components and wiring required for controlling a refrigeration system and displaying the temperature on a LCD screen is well known by one skilled in the art and is not considered part of this invention. Therefore, the schematic of FIG. 11 is used for general illustration purposes only.

The flow or pressure controller (82) is used to set the desired flow rate of the cooled liquid through the liquid circuit. The preferred flow rate is between one to four liters per minute (1-4 lpm). The flow controller (82) is also electrically coupled to the microprocessor controller (94) which is in turn electrically coupled to pressure sensors (122) and the LCD screen (84). The pressure sensors (122) are disposed between the liquid pump (68) and the supply port (72) on the supply line (106), and the return port (70) and the reservoir (66) on the return line (104). If the pressure exceeds or falls below a predetermined pressure, the microprocessor controller (94) will shut down the pump motor (69) and set off an alarm condition. The necessary mechanical and electrical components and wiring required for monitoring and controlling pump operation, line pressure, and displaying the pressure or flow rate on a LCD screen is well known by one skilled in the art and is not considered part of this invention. Therefore, the schematic of FIG. 11 is used for general illustration purposes only. Additionally or alternatively, a sensor, component or monitor powered via the rotating coupling 52 (see FIG. 7) may be used to provide feedback to the therapy system to adjust, regulate or control the chiller unit 10. The sensor, component or monitor may provide information regarding the mammal being treated or about the operation of the therapy system.

In operation, the heat exchange bladder or appliance (19, 20 or 21) is secured to the body portion of the animal to be treated by the fastener strips (29). Flexible return and supply conduits (17 and 18) are connected to the respective connectors (32 and 34) on the heat exchange bladder or appliance (19, 20 or 21) and ports (70 and 72) on the liquid control station (10). The liquid reservoir (66) is filled with potable water or the propylene glycol solution as previously discussed. The main power switch (76) of the control station (10) is switched to the on position. The main power lamp (116) lights signifying the liquid control station (10) is in operation. The microprocessor controller (94) monitors the temperature setting and the temperature of the liquid in the reservoir (66). If the liquid is warmer than the desired temperature setting, the microprocessor controller (94) will activate the compressor motor (92) which circulates the refrigerant through the condenser (86) and evaporator coils (88) thereby cooling the solution in the liquid reservoir (66). When the desired liquid temperature is reached as displayed on the LCD screen (84), the microprocessor controller (94) activates the liquid pump motor (69). The cooled water solution is drawn through the supply line (106) and the check valve (120). The cooled solution, then travels through the supply port (72) into the supply conduit (18). The cooled solution enters the heat exchange bladder or appliance (19, 20 of 21) through the supply connector (32). The cooled water is circulated through the flow channels (30) and exits the heat exchange bladder or appliance (19, 20 or 21) through the return connector (34). The water is forced through the return conduit (17) into the return port (70) of the liquid control station (10) and returns to the liquid reservoir (66) via the return line (104) where it is chilled and recirculated.

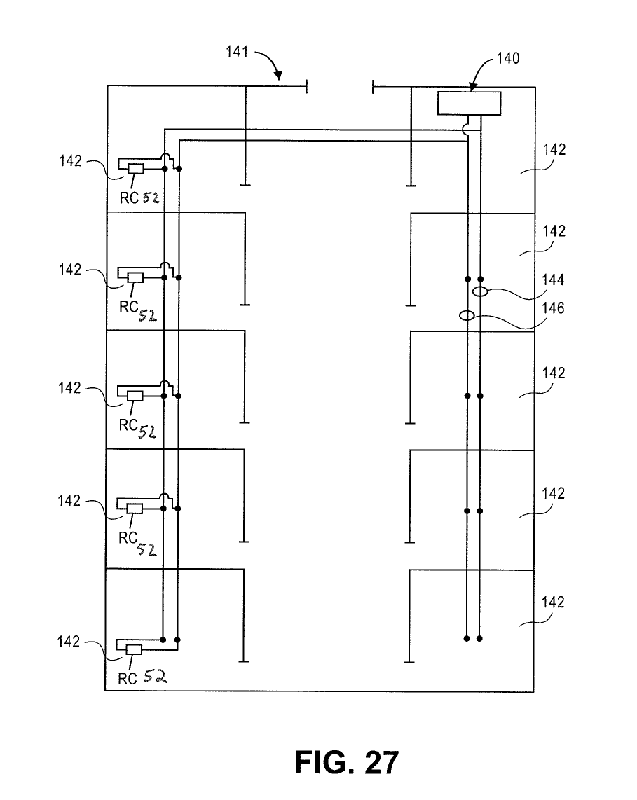

The supply and return ports (72 and 70) of the liquid control station (10) may be equipped with a manifold device to enable more than one pair of fixed side return and supply conduits (17 and 18) to be connected to the liquid circuit thereby enabling more than one rotating coupling 52 (and associated heat exchanger bladders or appliances 19, 20 or 21) to be connected to the control station (10) at one time. One exemplary manifold device is capable of receiving five pairs of return and supply conduits (17 and 18) from five rotating couplings 52. Such configuration may be useful in a multiple animal treatment scenario as shown in FIGS. 27, 28 and 29. It should be understood that the manifold device described above is for illustration purposes only and that a manifold device having more or fewer apertures can be constructed, depending upon the specific needs of a therapy system.

It should also be understood, that to enable multiple rotating couplings 52 as well as heat exchange bladders or appliances (19, 20 or 21) to connect to a single control station (10), a variable speed or variable pressure liquid pump (68) is required to ensure constant uniform flow and pressure to each appliance (19, 20 or 21) connected to the system. It should be appreciated that as more appliances (19, 20 or 21) and rotating couplings 52 are connected to the control station (10), more liquid must be circulated through the system. Likewise, as rotating couplings 52 or appliances (19, 20 or 21) are removed from the system, less liquid must be circulated through the system, thus changing the flow and pressure requirements of the pump (68).

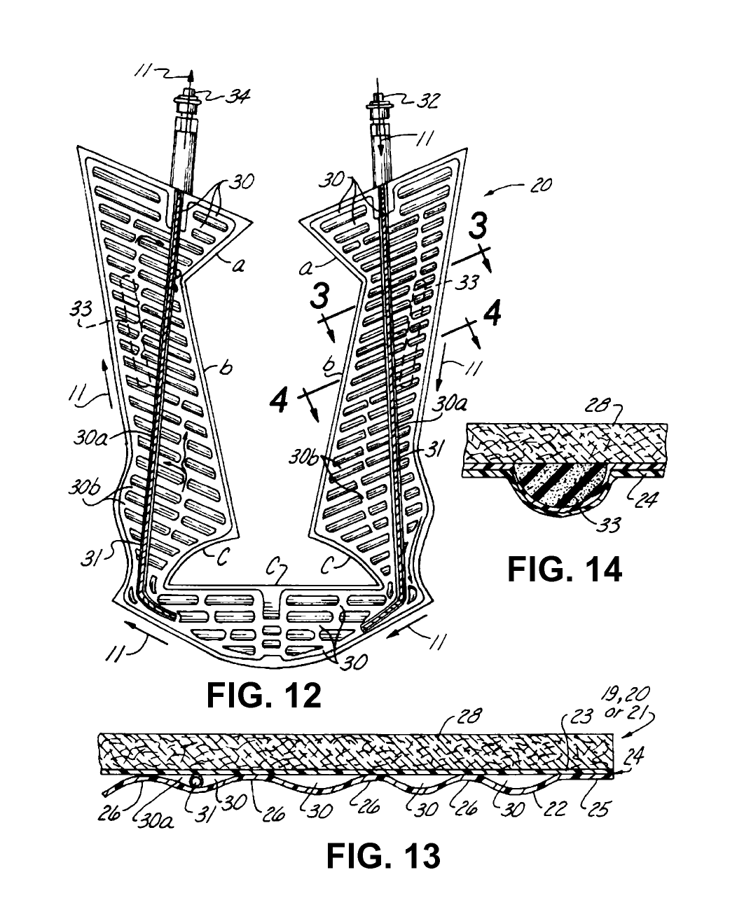

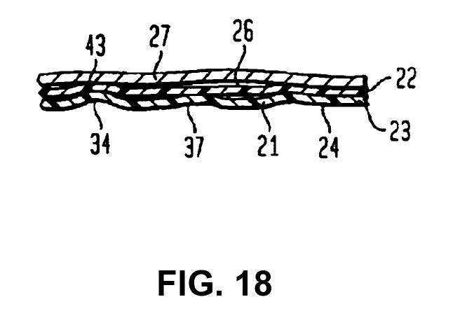

In general, a heat exchange bladder (also referred to as a wrap or an appliance) contains a plurality of fluid channels in a shape suited to for application to a portion of a mammal to receive therapy. Heat exchange fluid at the temperature desired for a given therapy is circulated through the heat exchange bladder in order to provide the indicated thermal therapy. Heat exchange bladders may be used without an accompanying air bladder as shown in FIGS. 12-17. Alternatively, the heat exchange bladder may include an air bladder that inflates during therapy to apply controllable pressure (i.e., compression) to the mammal. Examples of heat exchange bladders with both liquid and air bladders are described in FIGS. 18, 19, 20. It is to be appreciated that the shape, size, contours and attachment mechanisms of the exemplary heat exchange bladders shown and described in FIGS. 12-19 may be modified from the illustration in order to suit the needs of a specific therapy location of a mammal being treated. While described for treatment of a hoof in most cases, other treatment sites such as the back, chest or legs are also within the scope of the embodiments of the present invention.

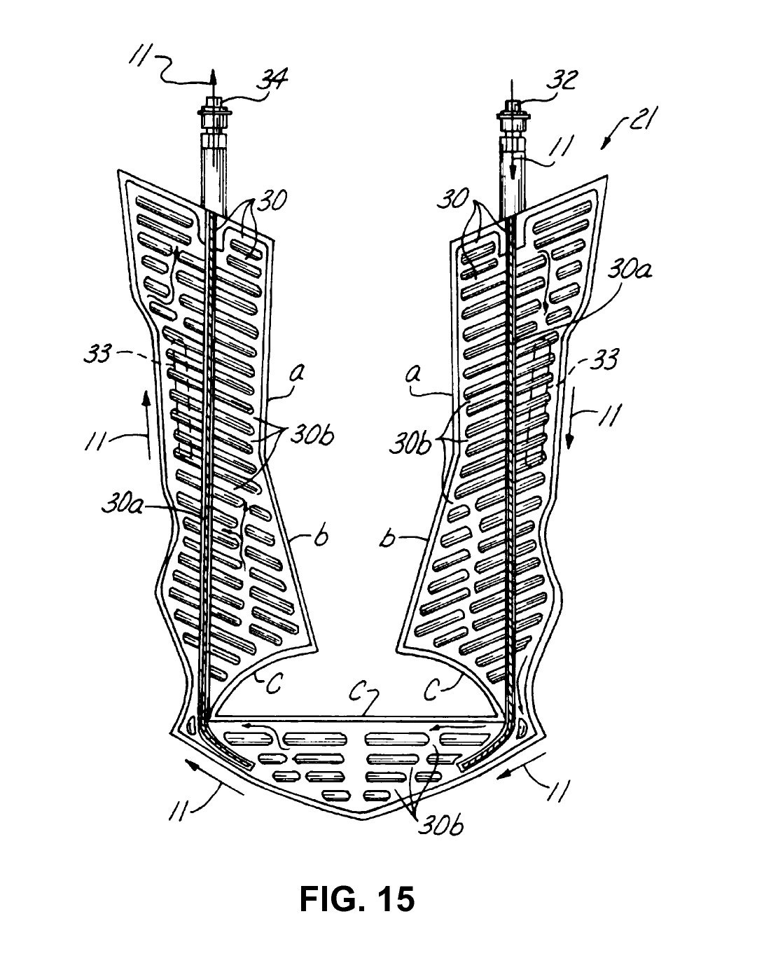

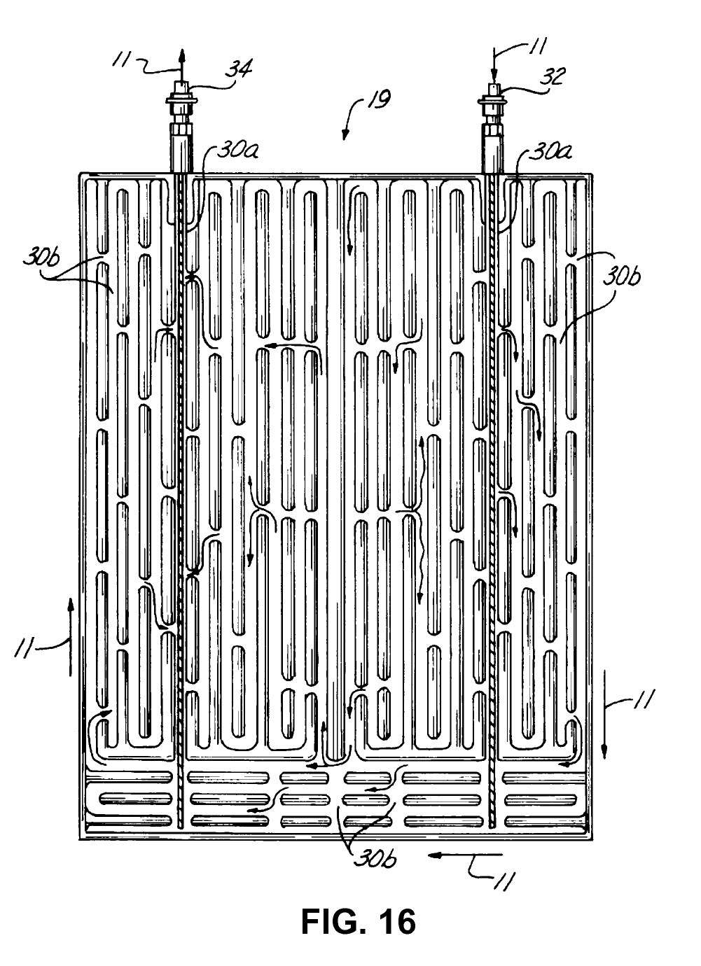

FIG. 12 shows a detailed flat layout view of the sealed envelope of a heat exchange bladder or appliance sized and shaped for a horse's leg. FIG. 15 shows a detailed flat layout view of the sealed envelope of a heat exchange bladder or appliance for a horse's front leg. FIGS. 12 and 15 are flat layout views of the front and rear leg appliances (21 and 20) respectively. FIG. 16 shows a detailed flat layout view of the sealed envelope of a heat exchange bladder or appliance for a horse's back. FIG. 16 is a layout view of the heat exchange bladder or appliance (19) for treatment of the horse's back (13). These figures will be discussed in further detail later.

FIG. 13 shows a sectional view of the heat exchange bladder or appliance taken along lines 3-3 of FIG. 12. As best illustrated in FIG. 13, which is a cross-sectional view taken along lines 3-3 of the rear leg appliance (20) of FIG. 12, but is typical of all appliances (19, 20 and 21), the heat exchange bladder or appliance is comprised of an inner wall (22) and outer wall (23). The walls (22 and 23) are constructed out of a water-impervious, synthetic, rubberized, polymeric material capable of remaining flexible and resilient at temperatures between 0 degrees Fahrenheit and 100 degrees Fahrenheit. The thickness of the walls (22 and 23) is not critical, though a thickness of about 4 mils to about 15 mils is preferred. The walls (22 and 23) are die-stamped or heat stamped together thus creating a single unitary sealed envelope (24) having a periphery seal (25) and a series of internal seals (26) thereby forming internal flow channels (30) between the two walls (22 and 23). The envelope (24) is bonded to an insulating jacket (28) thereby creating a single unitary insulated appliance (19, 20 or 21).

The insulating jacket (28) is preferably a closed cell synthetic polymeric foam. Other insulating materials such as open cell polymeric foams and fibrous composites may also be used. An insulating jacket thickness of about 100 mils to about 250 mils is preferred for optimum insulation and pad flexibility. An additional outside layer of washable, durable fabric such as nylon may be used to protect the insulating jacket from wear or from being soiled.

FIG. 14 is another sectional view of the heat exchange bladder or appliance taken along lines 4-4 of FIG. 12. As shown in FIG. 14, disposed between the outer wall (23) and the insulating jacket (28) are positioned a pair of conforming members (33), preferably constructed out of a polyurethane foam. The conforming members (33) are disposed in the appliances (20 and 21) between the outer wall (23) and the insulating jacket (28). These conforming members (33) act to conform the appliances (20 and 21) to the areas of concavity of the legs (14 and 15) between the cannon bone and the tendons to ensure substantial surface area contact of the appliance (20 and 21) with the legs (14 and 15).

Referring now to FIGS. 12, 15 and 16, in viewing the internal flow channels (30) of the respective appliances (19, 20, or 21), it should be appreciated that the flow channels (30) include larger main flow channels (30a) which branch throughout the appliance into a series of smaller branch channels (30b). These main channels (30a) and (30b) cover the entire surface area of the heat exchange bladder or appliance. It should be appreciated that the heat exchange bladder or appliances (19, 20 or 21) have a generally U-shaped flow configuration as illustrated by the arrows (11). A supply connector (32) and an return connector (34) in communication with the internal flow channels (30) are positioned on opposite sides of the U-Shaped flow configuration thus requiring the cooled liquid to enter the heat exchange bladder or appliance (19, 20 or 21) through the supply connector (32) on one side and circulate through the entire U-shaped flow channel (30) before exiting through the return connector (34) on the other side of the heat exchange bladder or appliance (19, 20 or 21).

It has been determined that the flow channels (30) may collapse due to the suction force of the liquid pump (discussed later), thereby preventing the cooling solution from dispersing throughout the internal flow channels (30) of the appliance (19, 20 or 21). To overcome this problem, spiral tubes (31) (see e.g., FIG. 13) are placed in the main flow channels (30a) to prevent them from collapsing. If the main flow channels (30a) cannot collapse, the cooled liquid will be able to circulate throughout the appliance do to the many alternate branch channels (30b).

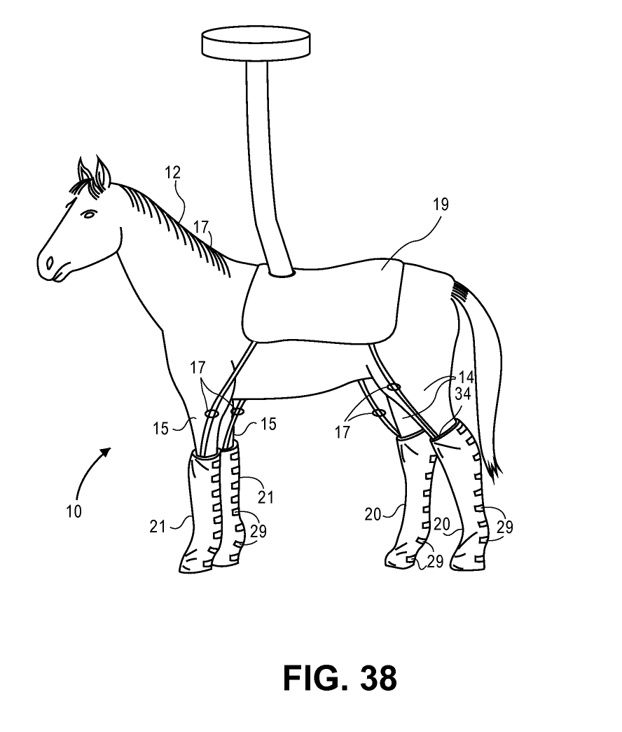

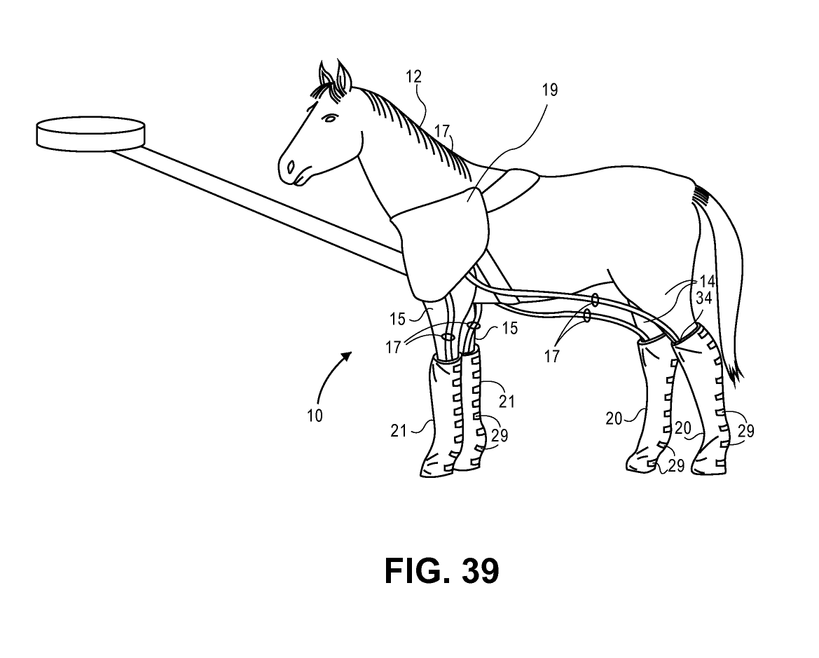

The heat exchange bladder or appliances (19, 20 and 21) are secured to the body portion to be treated by fasteners (29) (see FIGS. 38 and 39). The fasteners are preferably hook-type strips (29) (commonly referred to as hook and loop strips). When the heat exchange bladder or appliance (19, 20 or 21) is wrapped around the body portion to be treated, the hook fastener strips (29) are pulled and attached to the opposite side of the exterior of the insulating jacket (28) to secure the heat exchange bladder or appliance (19, 20 or 21) firmly in place. It should be appreciated that if the exterior surface of the insulating jacket (28) is of a type of material to which the hook-type fasteners (29) will not readily attach, loop-type strips (not shown) may have to be secured to the insulating jacket (28) for the hook fasteners (29) to attach to.

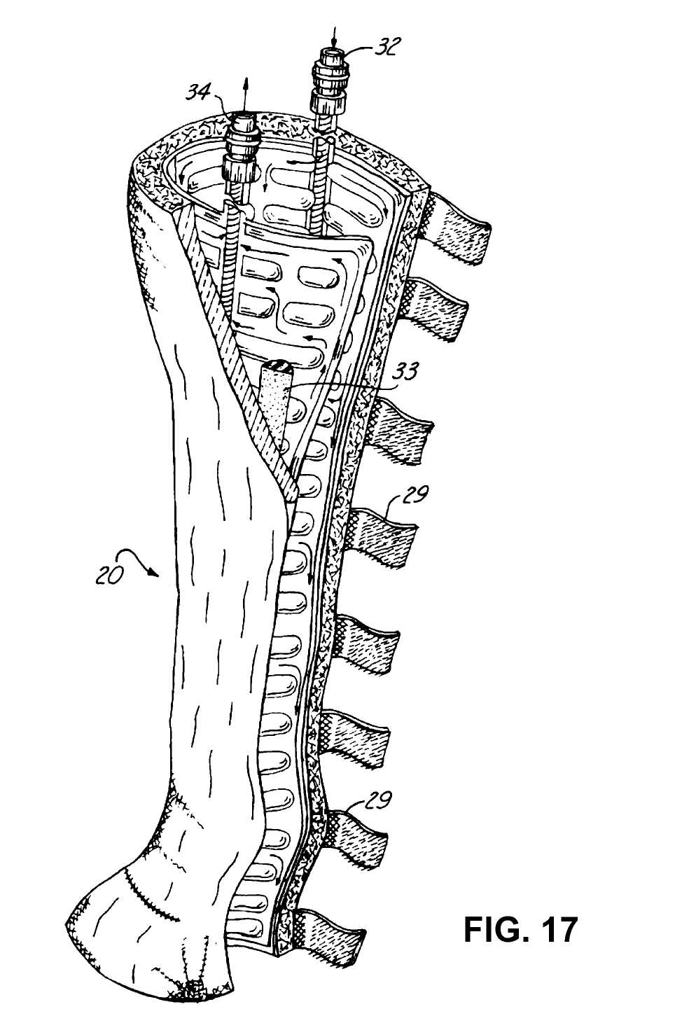

As mentioned previously, FIGS. 12 and 15 are flat layout views of the front and rear leg appliances (20 and 21) respectively. In order to construct the heat exchange bladder or appliances (20 and 21) from flat material, so that there is substantially complete surface area contact between the cooling surface of the heat exchange bladder or appliance and the body portion to be treated, the envelope (24) and insulating jacket (28) must have the general shape as shown in FIG. 12 or 15. It is desirable to construct the heat exchange bladder or appliances from flat material so that the envelope (24) can be die stamped or heat stamped as discussed above to create the internal flow channels (30) from two separate layers (22 and 23). Once the envelope (24) and insulating jacket (28) are fitted and bonded together, the edges a-a, b-b, and c-c (FIGS. 12 and 15) are secured together by glue or stitching or both. The assembled appliance (20 or 21) will then have the 3-dimensional configuration of the respective body portion to be treated, for example as shown in FIG. 17. FIG. 17 is a perspective view of a configured heat exchange bladder or appliance for a horse's rear leg.

Heat exchange bladders or appliances may also include a pneumatic side or air bladder to provide compression therapy during treatment. In cases where an air bladder style heat exchange bladder is used, the system includes an air source such as a pump or blower to controllably inflate the air side. Moreover, the rotating coupling is provided with appropriate fixed side and rotating side connections and ports to enable the air supply to be provided to via the rotating coupling.

FIG. 18 is a partial section view of a heat exchange bladder or appliance having both a fluid bladder and an air bladder. As best illustrated in the partial sectional view of FIG. 18, the operational layers include a compliant bladder 21 for containing the circulating heat exchange liquid. The bladder 21 may be of similar construction and operation as described above in FIGS. 12-17.

The bladder 21 is defined by a pair of generally parallel and liquid tight flexible, or in other words, compliant walls 22 and 23, which walls are sealed together by, for example, heat sealing as indicated at 24. The heat exchange bladder also includes a compliant gas pressure or air bladder 26 which overlays the heat exchange bladder as illustrated to direct gas (most simply, air) pressure against a body part in support of the thermal therapy. This compliant gas pressure bladder is also defined by a pair of generally parallel and flexible walls 22 and 27. In this connection, wall 22 is a common wall, i.e., one side of the same aids in defining the gas pressure bladder whereas the other side aids in defining the liquid bladder. Thus three compliant walls are all that is necessary to define the two separate bladders. Wall 27 is also secured at 24 via heat sealing with walls 22 and 23. Each of the walls 22, 23 and 27 is made of a nylon material suitably coated with a polyurethane to provide both the heat sealing qualities and the needed liquid or air impermeability.

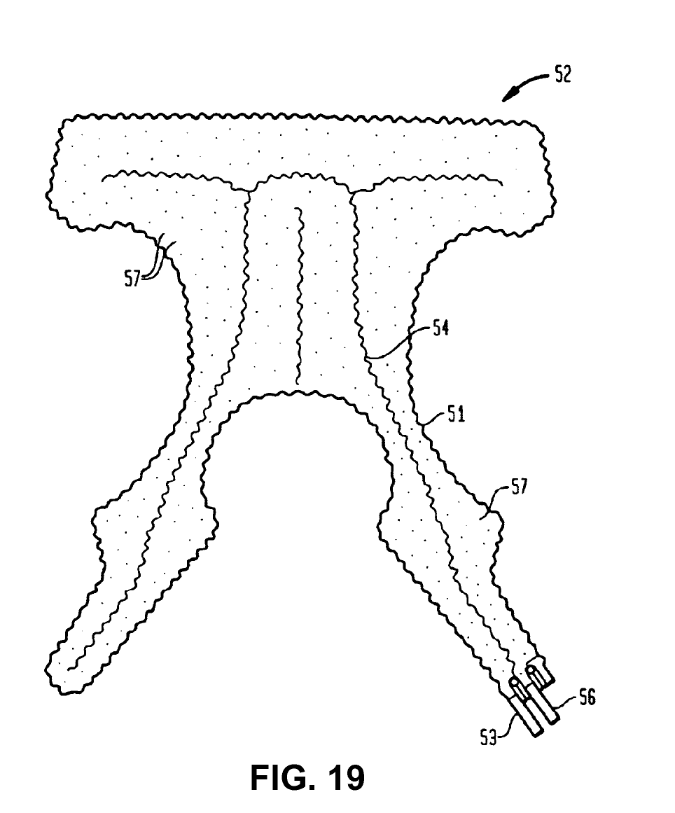

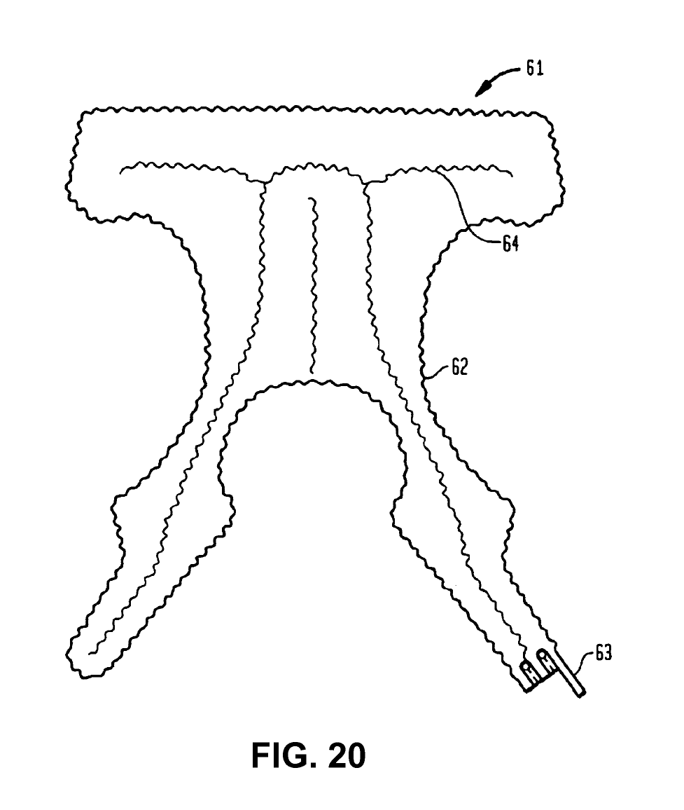

FIGS. 19 and 20 are views of a heat exchange bladder or appliance having on one side a fluid bladder (FIG. 19) and on the other side an air bladder (FIG. 20). FIGS. 19 and 20 show an arrangement for the bladders of the vest garment therapy component similar to that of the cap component. With reference to FIG. 19, the border 51 of the heat exchange bladder 52 is defined by curvilinear ripples. Liquid is introduced into the bladder via inlet tube 53, flows throughout the bladder along a path defined by fences 54, and exits through exit tube 56. A matrix of dot connections 57 is provided throughout the interior to disperse the liquid and assure that all aspects of the portion receiving therapy is subjected to the heat exchange liquid.

The interior of the gas pressure bladder 61 is represented in FIG. 20. The border 62 is defined by curvilinear ripples which register with those of the liquid bladder, and air pressure is introduced into the same via tube 63. Such gas pressure bladder includes a fence configuration 64 which acts as the connections within the bladder between the walls. This fence configuration registers with the fence configuration in the liquid bladder shown in FIG. 19, with both this configuration and the fences in FIG. 19 being simultaneously formed by heat sealing in accordance with an appropriate manufacturing process. In this connection, the sheets of material from which the two bladders of the heat exchange bladder or appliance are formed are heat sealable coated nylon. It is to be noted that in this arrangement, the dot connections are not also provided in the air pressure bladder.

It is contemplated that a selection of the dot connections can be made to provide a desired connection arrangement for a gas pressure bladder. Moreover, in some situations it may be desirable to use both dots and a portion of the fences in a liquid bladder to provide connections in the overlaying gas pressure bladder.

Additionally or alternatively, the heat exchange bladder may have the design shown in U.S. Design Application No. 29/329,007 entitled “Equine Hoof Wrap” (filed Dec. 5, 2008) and U.S. Design Application No. 29/267,593 entitled “Equine Back Wrap” (filed Oct. 16, 2006). Moreover, the heat exchange bladder may be configured as provided in any of the applications incorporated by reference or provide additional aspects such as those described in U.S. Pat. No. 6,695,872 and U.S. Patent Application Publication US 2010/0145421.

Torque Transmitting Hose Assembly

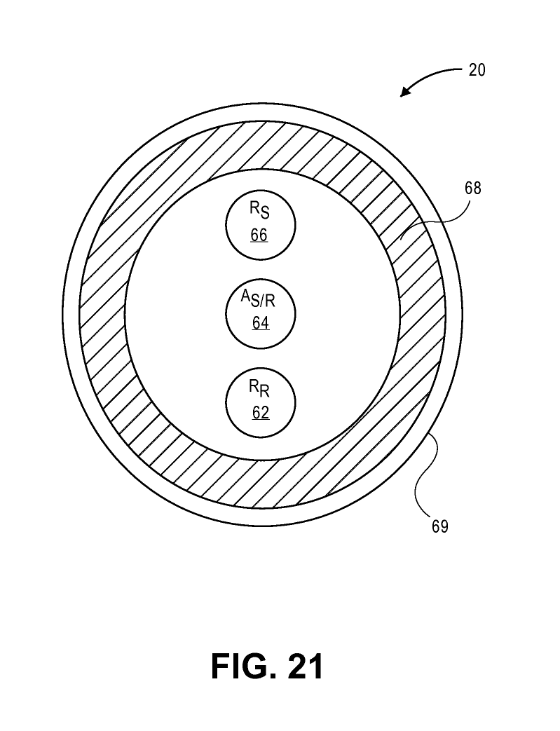

Another aspect of the invention is the hose 20 in FIG. 1. A cutaway view of the hose 20 is illustrated in FIG. 21. FIG. 21 is a section view of a conduit used to provide heat exchange fluid supply and return and air supply and return. The outer sleeve 68 of the hose 20 may include insulation material. The hose includes a refrigerant (or cooling) fluid supply line 66 and refrigerant (or cooling) fluid return line 62, and an air supply/return line. The refrigerating (or cooling fluid) may be of any type; however the use of non toxic fluids such as propylene glycol or BioGlycol is preferred.

One aspect of the present invention is the ability to transmit torque from the mammal (e.g., horse) as it walks in a circle, back to the rotating coupling. If a moment arm is not used, this torque is transmitted by the torsional stiffness of the hose 20. With a very soft hose, the torque will not be transmitted adequately back to the swivel axis. With too stiff of a hose, the hose will not be sufficiently flexible to be comfortable to the horse. Using an insulation material with desired stiffness (Armacel ½″ wall×1⅛″ Internal Diameter, for example) is one technique to achieve a good balance between flexibility and torsional stiffness. However, the torsional stiffness could be modified by adding another material around the outside of the hose to further transmit the torque. (See for example outer hose liner 69 in FIG. 21). Outer hose liner 69 may be, for example, formed from materials used for flexible electrical conduit. Other materials and constructions for hose 20 may be provided and may vary with the composition of the hoses 62, 64 and 66. In one aspect, the hose 20 may have a rotational stiffness of between about 1 in-lb/rad and 25 in-lb/rad. In one aspect, the rotational stiffness described herein describes the quality of a hose or hoses that connect the rotation coupling to a manifold (see FIGS. 22, 23, 30-39) or to an heat exchange bladder or appliance (see FIGS. 1-3).

FIG. 22 illustrates an embodiment of a thermal therapy system using having a spring biased pivoting overhead rotating coupling between the heating or cooling unit and the heat exchange bladders or appliances used to treat the front and rear legs or hooves of a horse. FIG. 23 illustrates an embodiment of a thermal therapy system using having a ram biased pivoting overhead rotating coupling between the heating or cooling unit and the heat exchange bladders or appliances used to treat the front and rear legs or hooves of a horse.

FIGS. 22 and 23 provide alternative rotating coupling supports or biasing elements. In contrast to the coil return used in FIG. 1 or the counterweight system of FIG. 2, the systems shown in FIGS. 22 and 23 each use a pivot, arm and biasing element arrangement to help support the weight of the rotating coupling 52, hose 20, manifold (if present) and associated hoses with each heat exchanger in use on the mammal. As shown in both FIGS. 22 and 23, the biasing element is connected to the rotating coupling 52. Other configurations are possible. Alternatively, the biasing element is attached to at least one of the hose connecting the heat exchange bladder supply conduit to the rotating side supply conduit and the hose connecting the heat exchange bladder return conduit to the rotating side return conduit.

In contrast to the arrangements of FIGS. 1 and 2 where the rotating coupling is attached to an overhead structure (FIG. 1) or suspended (FIG. 2), the rotating coupling 52 in FIGS. 22 and 23 are supported by an arm and pivot. The pivot is connected to the overhead support. The arm is supported at one end by the pivot and at the other by the biasing element. The biasing element may be any of a number of suitable alternatives that support the weight of the system and provide for movement of the animal as shown in phantom in FIGS. 22 and 23. The biasing element may be a counterweight system (FIG. 1), a spring loaded return coil (FIG. 2), a spring (FIG. 22) or a hydraulic ram and a pneumatic ram (FIG. 23).

As shown in FIGS. 22 and 23, at least a portion of the biasing element moves along with the movement of the mammal being treated by the treatment system. In these examples, biasing element extends and contracts as the support arm pivots about the pivot point/structure. These figures also illustrate placing a heat exchange bladder in fluid communication with a rotating coupling and the rotating coupling in fluid communication with a heating or chilling unit while circulating heat exchanging fluid from the heat exchanger through the rotating coupling to the heat exchange bladder while movement performed by the mammal rotates the rotating coupling. Moreover, the systems illustrated show displacing the rotating coupling as the mammal moves from a first position to a second position. In one aspect, when in the first position or the second position the mammal is in a sleeping position. FIGS. 22 and 23 illustrate the horse (in phantom) receiving treatment in a laying or sleeping position.

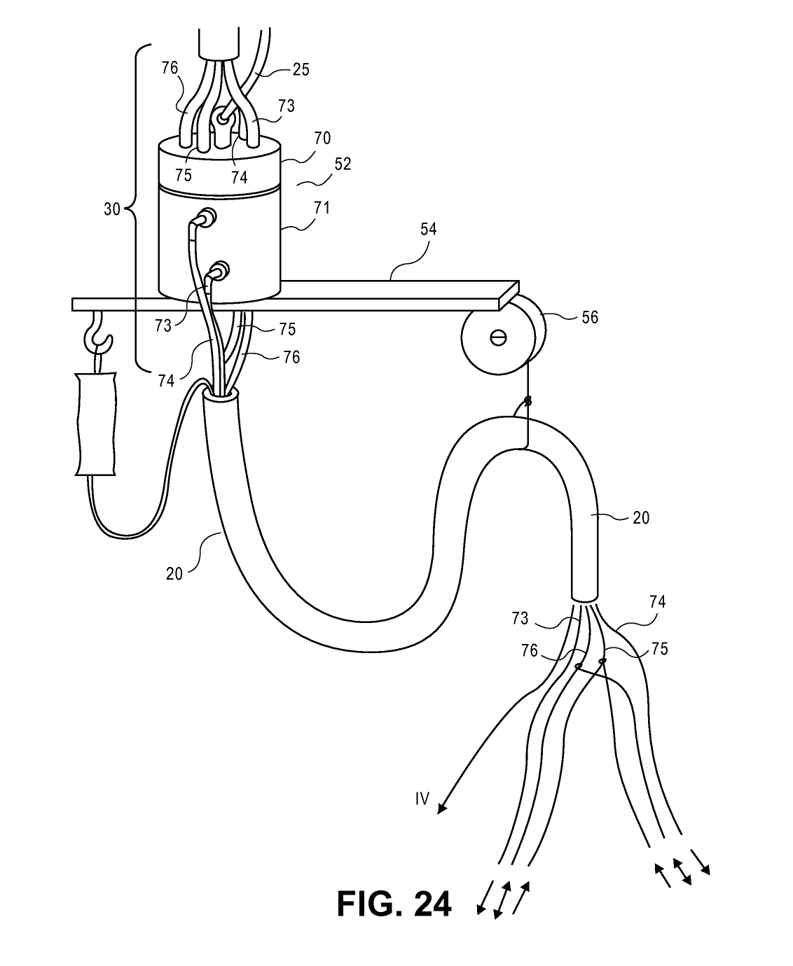

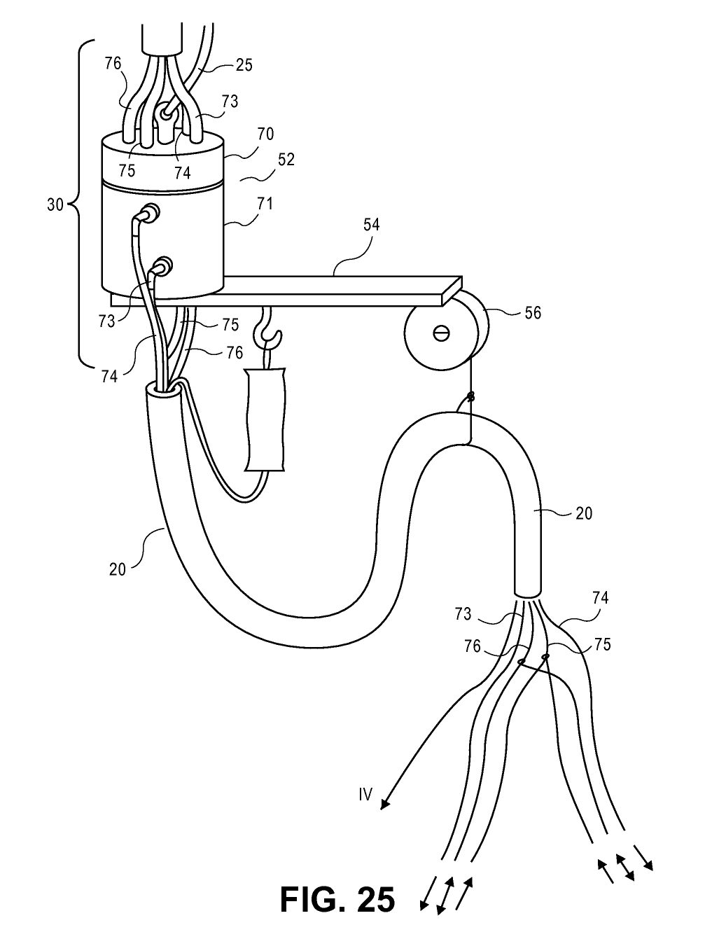

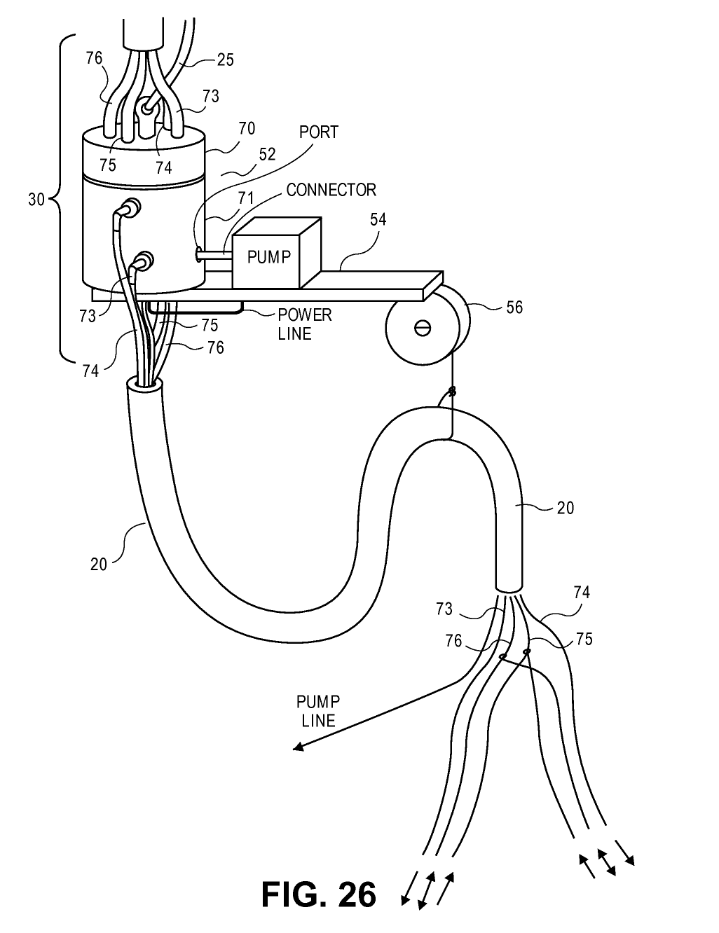

FIG. 24 is a perspective view of a rotating coupling of FIG. 4 that includes a fluid delivery bag supported along with the rotating coupling on the opposite side of the recoil mechanism and configured to provide the fluid to the mammal receiving therapy. In the example of FIG. 24, the container is an IV bag on an arm added in position either directly supported by rotating coupling or in position to counterbalance the hose and recoil assembly 56. FIG. 25 illustrates a container mounted on the rotating side of the rotating coupling on the same side as the biasing element (here, the recoil mechanism). There is provided a container (i.e., an IV bag for example) including a pharmacological agent and having a delivery conduit in communication with the mammal receiving therapy. Alternatively, the bag may be hung along the axis of the rotating coupling. The bag may also be located on the fixed side of the rotating coupling (i.e. outside the stall or treatment room) and the solution may be fed thru the rotating coupling in a separate channel than the cooling fluids. In some embodiments, the supply line connecting the bag to the mammal is provided along with the hose connected to the manifold (if used) or to the heat exchange bladder(s). The bag and associated delivery line may also be contained within the hose liner (see FIG. 21), attached to a hose or provided separately.

FIG. 25 provides an IV bag to support treatment of the mammal as in FIG. 24. FIG. 25 is a perspective view of a rotating coupling of FIG. 24 that includes a fluid delivery bag supported along with the rotating coupling on the same side as the recoil mechanism and configured to provide the fluid to the mammal receiving therapy. Unlike support configuration of FIG. 24, the IV bag in FIG. 25 is supported on the same support arm used to support the recoil mechanism 56. In this way, both the bag, the IV supply line, the recoil mechanism and the hoses are supported by the arm on the same side of the rotating coupling. It is to be appreciated that additional support structure useful in supporting the weight of the illustrated components is omitted so that the general arrangement of the components fed using the rotating side of the coupling 52 may be seen.

FIG. 26 is a perspective view of a rotating coupling of FIG. 24 that includes a pump and drug reservoir powered and controlled via the rotating coupling and supported on the same side as the recoil mechanism and configured to pump a fluid to the mammal receiving therapy. FIG. 26 illustrates a pharmacological agent in a container in fluid communication with rotating coupling to deliver the pharmacological agent to an outlet on the rotating side of the rotating coupling and a delivery conduit from the outlet on the rotating side to the mammal. In this example, there is a pump and drug reservoir mounted on the arm and connected to the rotating coupling as described above so that the drug or pharmacological agent enters the fixed side and is delivered to the rotating side and thence to the mammal receiving therapy. One or more conduits in the rotating coupling may be coated or treated to provide a sterile pathway from the drug pump and reservoir through the rotating coupling to the mammal receiving therapy. In addition, the pump is an example of a component that may receive power and/or control signals via electrical connection provided by the rotating coupling (see FIGS. 5 and 7). Alternatively, the pump may deliver the pharmacological agent without entering the rotating coupling and simply be powered and/or controlled by the electrical wires in the rotating coupling.

FIG. 27 is a plan view of a structure showing multiple stalls connected to a central liquid control station by piping for treating multiple animals at one time, including several stalls having rotating coupling type therapy connections. Another embodiment of the cold therapy system is shown in FIG. 27. Rather than having a portable liquid control station as described above, a central liquid control station (140) is used in a multi-stall barn (141), much like a central air conditioner in a building. In this embodiment, each stall (142) has access to a network of return and supply piping (144 and 146). In some of the stalls 142, the connections may be used for attaching at least one cold therapy appliance (19, 20 or 21). Some stalls 142 are equipped with a rotating coupling 52 as described herein. Mammals in the stalls with the rotating coupling may receive the therapy in conjunction with the benefits described herein such being able to move during therapy.

In addition, stalls 142 may be equipped with one or more of the improvements described above such as biasing elements (FIGS. 1, 2, 22, and 23) or drug delivery capabilities (FIGS. 24, 25 and 26), Thus, multiple horses in separate stalls (142) can be treated at one time. The network of return and supply pipes (144 and 146) terminate into a single return pipe and supply pipe which are then connected to the central control station (140). The central control station (140) is similar to the portable control station described above, except that a larger cooling reservoir (66), refrigeration system (64), and higher volume liquid pump (68) may be required depending on the number of animals, rotating couplings and heat exchange bladders or appliances expected to be used at one time.

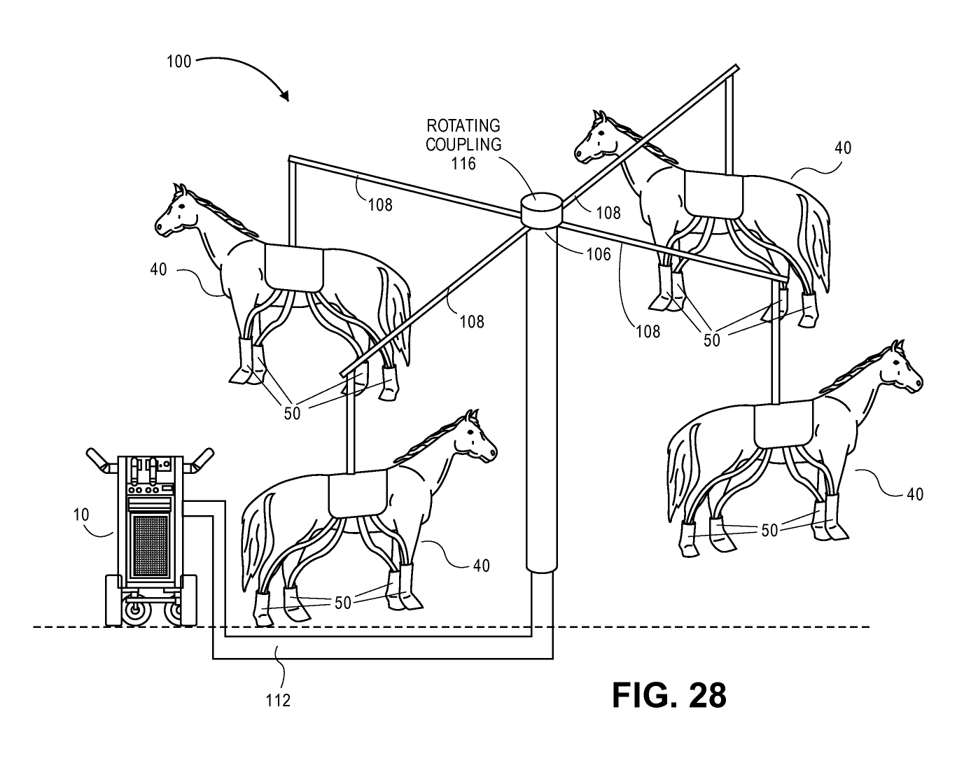

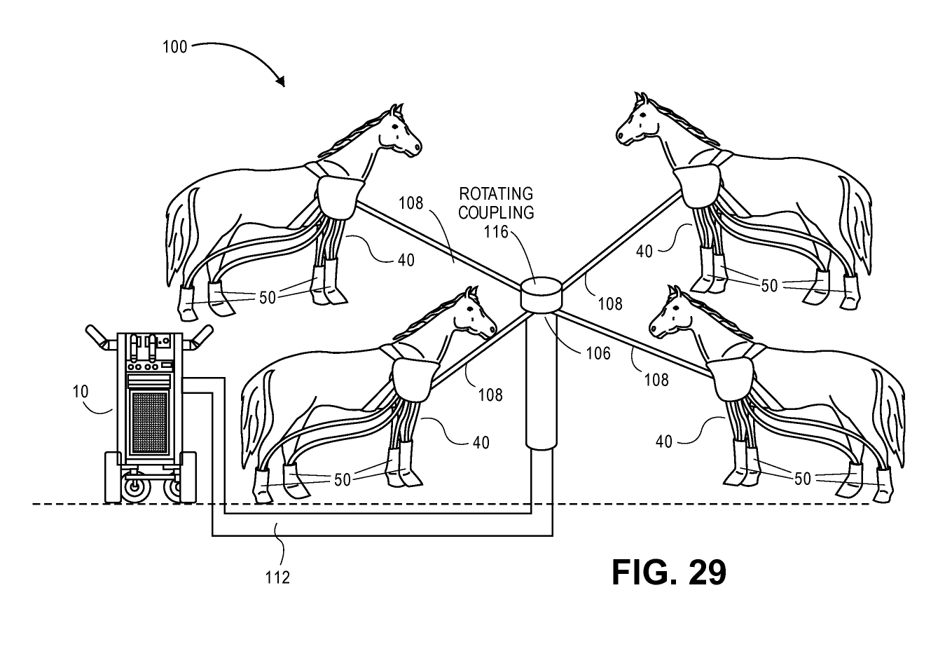

In still other alternatives, the therapy system having a rotating coupling may be combined with an exercise ring used to train and exercise horses. An exercise ring includes an upright or support that holds number of arms on a rotating coupling. A horse is hitched to an arm and allowed to walk about the upright. This conventional system is modified as shown in FIGS. 28 and 29. Four horses are shown receiving therapy from an upright that includes a rotating coupling 56 within the upright. The horse may exercise while receiving therapy. A horse may be receiving therapy using one or more of the heat exchange bladders as described herein in a stall 142 equipped for that purpose. The horse may be disconnected from the stall therapy system and walked out to the exercise therapy system of FIGS. 28 and 28. The horse would then have the manifold (shown in FIGS. 28 and 29) or alternatively the heat exchange bladders individually connected to the rotating coupling via appropriate connections on the arm 108.

FIG. 28 is a perspective view of a rotating coupling mounted on an upright to provide overhead connections to horses exercising in an arena while receiving therapy. FIG. 28 illustrates a thermal therapy system used in conjunction with an exercise arena. The chiller unit 10 may be a mobile unit as illustrated or a more permanent unit as in chiller 140 of FIG. 27. Similarly, the exercise therapy system may be connected to hoses as part of an installed chiller (FIG. 27) or outfitted with quick disconnect fittings to permit easy connection and use of a mobile chiller as shown in FIG. 28. The hose connections to the fixed side of the rotating coupling are provided through a hose(s) 112 underground as shown by the dashed line. Alternatively, the fixed side coupler supply and return lines may be provided above ground. The lines are used to supply heat exchange fluid and/or air as needed depending upon heat exchange bladder and type of therapy to be provided. As described above, the hoses 112 may be split using the rotating coupling into various outlet and return ports for delivery/return of coolant and/or air to one or more mammals 40. The hose 112 is connected to the fixed side of the rotating coupling 116 allowing movement of the mammal 40, especially circumferential movement along with the rotating coupling rotating side movement. Another coupling element 106 may be connected to the rotating coupling 116 and/or the hose 108/114 to allow movement of the hose 108/114 and the mammal 40. One or more mammals 40 may use the chiller unit 10 and simultaneously move in the arena. All the features previously described in the use of rotating couplings may be incorporated into FIG. 28.

As shown in FIGS. 28 and 29, there is an upright support connected to the rotating coupling along with another heat exchange bladder for another mammal. Here, there are additional heat exchangers connected to three additional manifolds (for a total of four) along with three other horses (for a total of four). The rotating coupling also includes another rotating side supply conduit, and another rotating side return conduit wherein the another rotating side supply conduit and the another rotating side return conduit are connected to the another heat exchange bladder. Additional conduits are provided depending upon the number of animals to be treated. In addition, a hose connecting the fixed side supply conduit to the heat exchange fluid supply conduit and a hose connecting the fixed side return conduit to the heat exchange fluid return conduit are within or along the upright support. In order to accommodate different size mammals or preferences for therapy, the upright height may be adjusted so that the system interfaces with the mammal at a desired height. The upright may be set to a height where the rotating coupling is above the head of the mammal (FIG. 28). Alternatively, the upright may be set to a height where the rotating coupling is at about chest height of the mammal or near chest height of the mammal (FIG. 29). The upright may be adjustable and the hoses within or along the upright configured to have sufficient slack and flexibility to accommodate changes in height. FIG. 29 is a perspective view of a rotating coupling mounted on an upright to provide chest height connections to horses exercising in an arena while receiving therapy.

Still other alternative therapy systems include the use of a manifold or distributor between the rotating coupling and the various heat exchange bladders being using to treat a mammal. One benefit of a manifold is that it may be configured to receive the conduits to and from the heat exchange bladders in a central location on the mammal. Additionally, rather than having each heat exchange bladder connected to individual ports on the rotating coupling, a single supply and single return port can be connected to the manifold to simplify attaching and detaching the mammal from the therapy system. Hoses connecting the manifold to the rotating coupling in a stall, for example as in FIG. 22, may simply be disconnected and then reconnected to the arena system as in FIG. 28. In this way, a manifold on the mammal helps organize the hoses and access to rotating coupling thereby simplifying the treatment provided utilizing a rotating coupling.

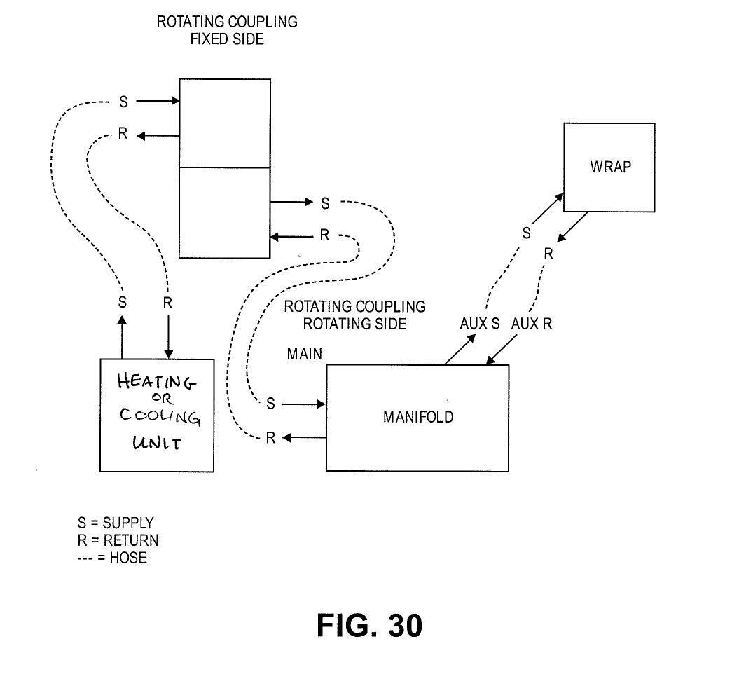

FIG. 30 is a schematic overview of a therapy system utilizing a manifold positioned between the rotating coupling and the one or more heat exchange bladders on a mammal receiving therapy. FIG. 30 is a schematic diagram of the various conduits used to connecting a heat exchange bladder, appliance or wrap via a manifold and rotating coupling to a heating or cooling unit. As shown in FIG. 30 there is a treatment system for a mammal having a rotating coupling having a fixed side supply conduit, a fixed side return conduit, a rotating side supply conduit, and a rotating side return conduit. The system includes at least one heat exchange bladder for the mammal being treated by the thermal treatment system. The heat exchange bladder has a supply conduit and a return conduit. There is a manifold having a main supply conduit, a main return conduit, at least one auxiliary supply conduit and at least one auxiliary return conduit. The system also includes a heating or chilling unit having a heat exchange fluid supply conduit and a heat exchange fluid return conduit (e.g., FIGS. 1, 9 and 10).

The system also includes a number of conduits or hoses to connect the various components and provide a fluid circuit. The hoses include: a hose connecting the manifold main supply conduit to the rotating side supply conduit; a hose connecting the manifold main return conduit to the rotating side return conduit; a hose connecting the at least one auxiliary supply conduit to the heat exchange bladder supply conduit; a hose connecting the at least one auxiliary return conduit to the heat exchange bladder return conduit; a hose connecting the fixed side supply conduit to the heat exchange fluid supply conduit; and a hose connecting the fixed side return conduit to the heat exchange fluid return conduit.

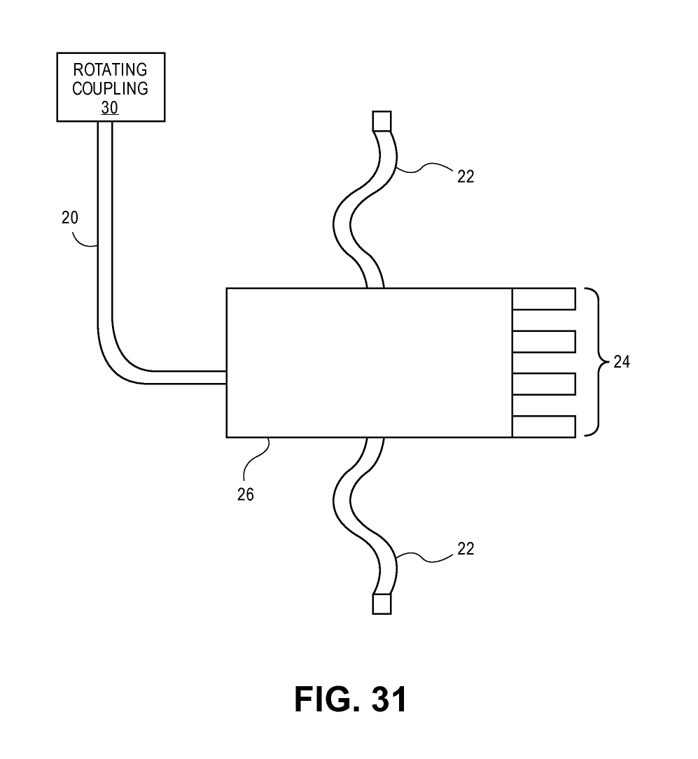

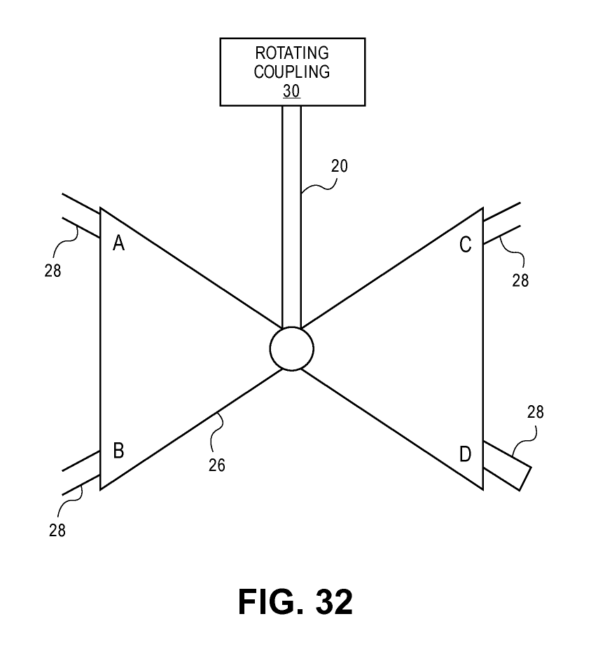

FIG. 31 is a perspective view of a manifold connected to an overhead mounted rotating coupling. FIG. 32 is a perspective view of a manifold connected to an overhead mounted rotating coupling. FIGS. 31 and 32 illustrate alternatives manifold configurations between the rotating coupling and the hoses attached to various heat exchange bladders. In FIG. 31, a manifold 26 configured to be placed over the spine of the mammal and then attached with straps 22. One or more connections 24 may provide cooling and/or compression to the heat exchange bladders (not shown). Each connection 24 may be connected to a different heat exchange bladder. As shown, the hose 20 connecting the rotating coupling to the manifold is shaped to connect to an overhead rotating coupling configuration (e.g., FIG. 1, 22 or 23). The hose 20 may be shaped to bend towards the rotating coupling as shown.

In FIG. 32, the manifold 26 is also configured to be placed over the spine of the mammal. In contrast to the manifold of FIG. 31 where all connections 24 were on one end of the manifold, the manifold of FIG. 32 connections 28 distributed to align more closely with the front and rear legs of the mammal. As with the other embodiments described herein, one or more connections 28 may provide a fluid conduit for heat exchange fluid and/or air for operation of a heat exchange bladder. The shape of the manifold 26 in FIG. 32 is configured to simplify conduit connections between heat exchangers on each leg of a mammal and the manifold. Each connection 28 may be connected to a separate heat exchange bladder. In contrast to FIG. 31, the connecting hose between the rotating coupling and the manifold in FIG. 32 is centrally located in the manifold rather than the end. Similar to the earlier embodiment, the hose 20 is directed upward towards the rotating coupling as in the overhead rotating coupling situations discussed above. While single connections are shown in FIGS. 31 and 32, it is to be appreciated that additional connections may be added or the connection may be either single, double or multiple hose connections at the various manifold locations.

FIG. 33 is still another alternative manifold configuration. FIG. 33 is a perspective view of a manifold connected to an overhead mounted rotating coupling with pivoting connections to four heat exchange bladders or appliances. FIG. 33 is a manifold similar to the manifold of FIG. 31 in that there is an end manifold connection and that the supply connection is shaped to extend from the manifold toward the rotating coupling (i.e., the hose is curved so that it will accommodate the orientation between a spine mounted manifold and an overhead mounted rotating coupling). In contrast to FIG. 31, the connection points for the heat exchange bladders are arranged as in FIG. 32 to more closely correspond to treatment sites on the leg or hoof.

Optionally, the manifold connection points are provided with rotating couplings that permit movement of the hose(s) connected to the heat exchange bladder. The use of a movable coupling between the manifold and the heat exchanger may help mitigate movement of the manifold as the animal moves during therapy. A moveable coupling may be used between the manifold and the hose to heat exchange bladders as shown in FIG. 33.

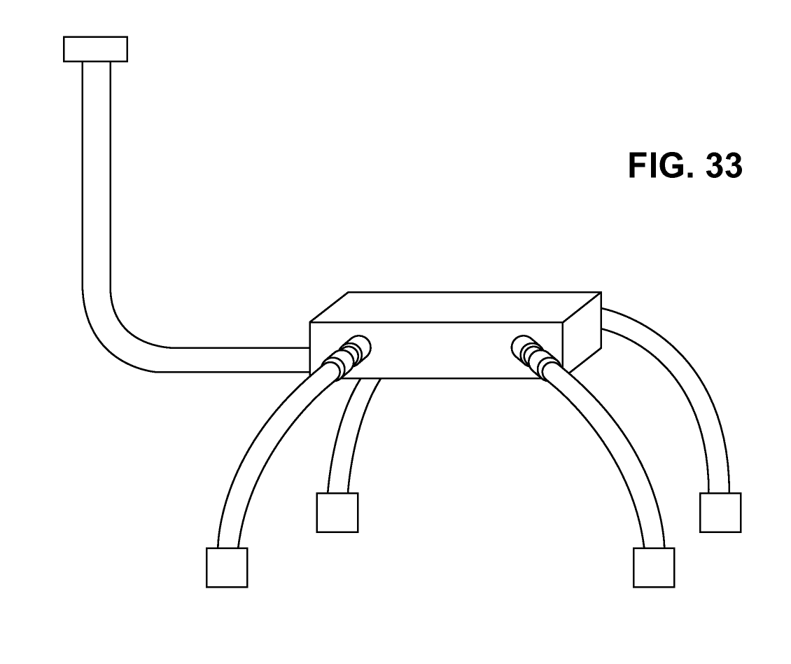

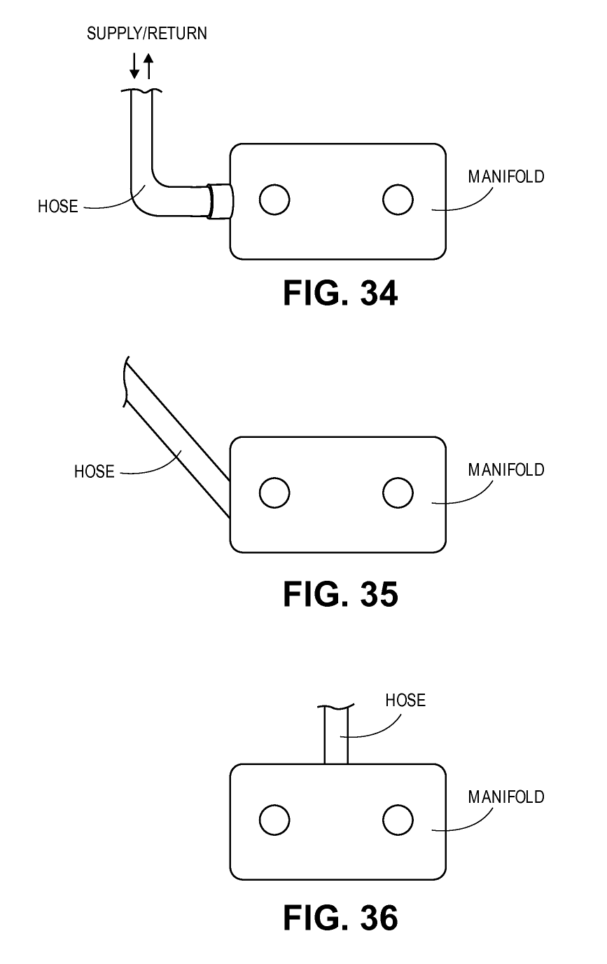

A moveable coupling may be used between the hose leading to the rotating coupling and the manifold as shown in FIG. 34. FIG. 34 is a perspective view of a manifold connected to an overhead mounted rotating coupling having a pivoting or rotating connection on the manifold. In still other alternative manifold configurations, the manifold inlet/return used to connect to the rotating coupling may be angled to accommodate the orientation between the manifold and the rotating coupling. The coupling in FIG. 35 shows an inclined angle that would be useful when an overhead rotating coupling is used (i.e., FIGS. 1, 23, 24 and 38). FIG. 35 is a perspective view of a manifold connected to an overhead mounted rotating coupling with a manifold connector angled towards the orientation of the rotating coupling. Alternatively, the angle may be flatter or approaching a nearly horizontal orientation if the rotating coupling is located at or near chest level (i.e., FIGS. 29 and 39).

FIG. 36 is a perspective view of a manifold having a connection directed towards an overhead mounted rotating coupling. FIG. 36 is an end view of a top fed manifold similar to the manifolds of FIGS. 31 and 33. FIG. 36 illustrates a central top mount connection point between the manifold and an overhead rotating coupling.

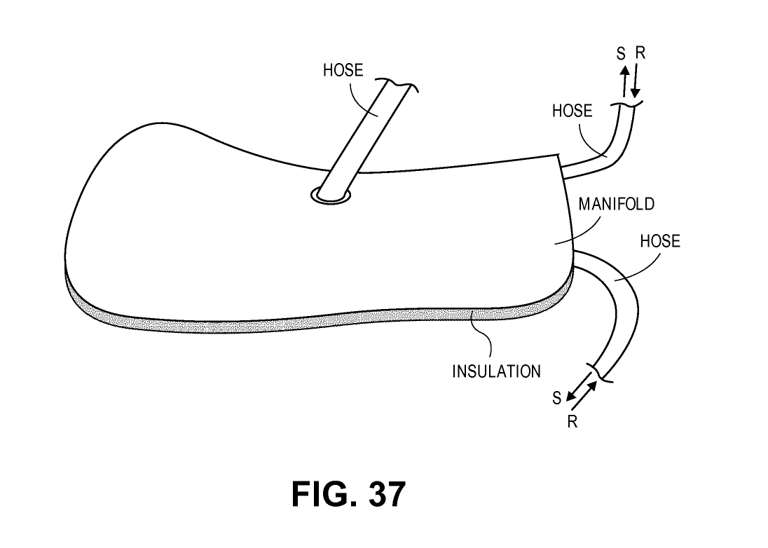

FIG. 37 is another example of a top fed manifold. FIG. 37 is a perspective view of a manifold shaped for placement on a mammal having an insulated layer and a moveable top mounted connection directed towards an overhead mounted rotating coupling. In the manifold embodiment of FIG. 37, connection ports for individual heat exchange bladders as provided as shown for easy access to hoof and leg therapy locations as described above with regard to FIGS. 32 and 33. Additionally, the connection point for the supply hose at the upper manifold surface may also include a swivel or moveable connection as described above. The manifold may also have a curved shape to accommodate the portion of the mammal supporting the manifold. In the illustrated embodiment, the manifold is saddle shaped to conform to the back of a horse (see also the manifold shapes of FIGS. 22, 23, and 28, for example). Alternatively, the manifold may be shaped to conform to the chest of the mammal as shown in FIGS. 29 and 39, for example. In addition, insulation is provided on the surface of the manifold contacting the mammal. The insulation will help with heat transfer efficiency as well as enhanced comfort for the animal receiving therapy. The insulating layer may be attached to and be a part of the manifold or may be a separate layer that is placed between the manifold and the mammal. The insulting material is formed from any suitable insulating material such as, for example, ½″ EPDM foam.

FIGS. 38 and 39 illustrate manifold based therapy systems. FIG. 38 illustrates an embodiment of a thermal therapy system using having an overhead mounted rotating coupling connected to a back mounted manifold that is connected to four heat exchange bladders or appliances used to treat the front and rear legs or hooves of a horse. FIG. 39 illustrates an embodiment of a thermal therapy system using having a chest level mounted rotating coupling connected to a chest mounted manifold that is connected to four heat exchange bladders or appliances used to treat the front and rear legs or hooves of a horse.

In these illustrative embodiments, the manifolds have been formed to conform to a part of the mammal receiving therapy. In addition, the ports used to connect the heat exchange bladders are positioned on the manifold to make connections easier. In other words, the connections ports are provided in the manifold in positions that assist in the connection of heat exchange bladders from leg or hoof therapy sites.

FIGS. 38 and 39 illustrative alternative manifold configurations. Features and design aspects of the manifold embodiments described herein may be modified to accommodate the relative orientation/location of the rotating coupling as well as being shaped to conform to the portion of the mammal that supports the manifold. FIG. 38 illustrates a manifold embodiment configured for interaction with an overhead rotating coupling while being borne on the back of a mammal. FIG. 39 illustrates a manifold embodiment configured for interaction with a chest level mounted rotating coupling while being borne on the breast of the mammal.

FIG. 38 illustrates an embodiment of a manifold configured to operate with an overhead rotating coupling. Heat exchange bladders are shown being used to treat body parts 14 and 15, for example, of a horse 12. Although described and illustrated herein as being particularly designed for the treatment of a horse's legs, it should be understood that the manifold and heat exchange bladders may be adapted for the treatment of other body parts of various animals including humans. Therefore the scope of the aspects of the invention described herein should not be considered as limited only to the treatment of horses.

The back mounted manifold based therapy system is connected between the portable liquid control station 10 and each of the heat exchange bladders or appliances 19, 20 or 21 by a rotating coupling. In the embodiment of FIG. 38, the rotating coupling is an overhead mounted rotating coupling. In one aspect, the various fluid connections are provided by a pair of insulated, flexible, liquid supply and return conduits (18 and 17 respectfully). Depending on system operating parameters, the supply lines may be from ⅜ to ½ inch in diameter. The heat exchange bladder or appliance 19, 20 or 21 is configured for the body portion of the animal to be treated in a manner which provides substantially total surface contact. As shown in FIG. 38, the heat exchange bladder or appliance 19, 20 or 21 may be configured to receive the back 13 of a horse 12 which encompasses the withers, the back, the loin, and the croup or alternatively the lower rear leg 14 of a horse 12 which encompasses the hock, cannon, ankle, pasterns and hoof, or alternatively, the lower front leg 15 of a horse 12 which encompasses the knee, cannon, ankle, pasterns and hoof.

FIG. 39 illustrates a chest mounted manifold based therapy system. The chest mounted manifold illustrated in FIG. 39 is similar to the back mounted manifold in FIG. 38 in many ways. One difference is that the shape of the manifold is changed to accommodate the shape of the chest of the mammal rather than the back of the mammal. In addition, the placement of the connection ports between the chest mounted manifold and the heat exchange bladders are also distributed and oriented to aid in connecting the heat exchange bladders (located on the legs in the illustrated embodiment) to the chest mounted manifold.

The thermal therapy system described herein allows new ways of providing therapy to mammals by enabling the animal to move or even exercise during therapy. Since many therapies last for one or more days, the ability of the animal to remain active is thought to help promote healing and improve the therapy outcome. There are provided improved methods of providing therapy to a mammal. In one aspect, there is a step of attaching a heat exchange bladder to a portion of the mammal. Next, placing the heat exchange bladder in fluid communication with a rotating coupling and the rotating coupling in fluid communication with a heating or chilling unit as discussed above. Thereafter, therapy is provided by circulating heat exchanging fluid from the heat exchanger through the rotating coupling to the heat exchange bladder. Additionally, the therapy continues while movement performed by the mammal rotates the rotating coupling.

In still additional aspects of the improved method of providing therapy, there is the step of displacing the rotating coupling as the mammal moves from a first position to a second position. In one aspect, the first position or the second position the mammal is in a sleeping position. In still another aspect, the therapy method includes extending a biasing element during the displacing step when the rotating coupling is displaced about a pivot point connected to the rotating coupling. Various biasing elements are illustrated and described above with regard to FIGS. 1, 2, 4, 22, and 23. Pivoting supports are also illustrated in FIGS. 22 and 23. Still further, the method may include providing therapy whereby the displacing step includes displacing the rotating coupling about a pivot point, such as shown in FIGS. 22 and 23.

The method of providing therapy may also include, during the circulating step, maintaining the rotating coupling above the head of the mammal receiving therapy. Examples of systems that maintain this height are shown in FIGS. 1, 2, 22, 23 and 28. Alternatively, the rotating coupling may be maintained at about chest height during therapy as shown in FIG. 29.

Still further, in another aspect of the invention, the method of providing therapy includes performing the attaching step, the placing step and the circulating step with another mammal such that circulating step is performed using one rotating coupling. Examples of this method are illustrated and descried with regard to the exercise arena embodiments of FIGS. 28 and 29. Also exemplified by the arena is the method of moving the mammal and the another mammal in a generally circular path about the rotating coupling during the circulating step.

Still further alternatives are possible.

The various embodiments of conduits, heat exchangers and/or manifolds described herein may also be designed to prevent leakage of the coolant or heat exchange fluid. These embodiments also provide easy access to hook/unhook the chiller unit 10 or other associated component using a quick disconnect couplings, for example. The glycol solutions used in some systems are sticky and hard to wipe up when spilled. Standard liquid connectors allow drips during disconnection. Using connectors that are drip free during connection and disconnection is desirable.

Other embodiments and systems described herein may also use quick disconnect fittings such as for example in the single stable of FIGS. 1 and 2, the multiple stalls FIG. 27 or in the arena of FIGS. 28 and 29. In one aspect, a mobile chiller (FIG. 1, 2, 9, 10, 28 or 29) may be used and connected to the arena when needed. Alternatively, the arena could be plumbed to a main unit as in the multi-stall that has a main heater chiller unit (see multi-stall FIG. 27).

Another aspect of the invention comprises adding insulation (or anti-freezing agent, fluid or material) to the rotating coupling. The rotating coupling or rotary union may particularly have insulation and or coating to prevent ice formation.

Yet another aspect of this invention is to add a flavor additive and/or odor to the chiller coolant or fluid so that the horse would not be tempted to lick any spilled coolant. The coolants used are typically sweet, and sometimes toxic. Adding a bad flavoring or odor to the coolant will discourage the animals from drinking any spilled coolant.

Another aspect of the invention is to provide a method of preventing soiling of the hose, and/or preventing the spread of disease. Adding a disposable liner around the hose may be provided for this purpose.

While several embodiments of the present invention have been shown and described herein, such embodiments and alternatives are provided by way of example only. Numerous variations, changes, and substitutions are possible and within the scope of the various aspects of the invention. The various alternatives to the embodiments of the invention described herein may be employed in practicing the invention. The following claims further detail the methods and structures within the scope of the various alternatives, embodiments and their equivalents to be covered thereby.

What is claimed is:

1. A treatment system for a mammal, comprising:
a rotating coupling having a fixed side supply conduit, a fixed side return conduit, a rotating side supply conduit, and a rotating side return conduit;
a heat exchange bladder for the mammal being treated by the thermal treatment system, the heat exchange bladder having a supply conduit and a return conduit;
a heating or cooling unit having a heat exchange fluid supply conduit and a heat exchange fluid return conduit;
a hose connecting the heat exchange bladder supply conduit to the rotating side supply conduit;
a hose connecting the heat exchange bladder return conduit to the rotating side return conduit;
a hose connecting the fixed side supply conduit to the heat exchange fluid supply conduit; and
a hose connecting the fixed side return conduit to the heat exchange fluid return conduit.

2. The treatment system for a mammal according to claim 1 further comprising:
a biasing element connected to the rotating coupling or at least one of the hose connecting the heat exchange bladder supply conduit to the rotating side supply conduit and the hose connecting the heat exchange bladder return conduit to the rotating side return conduit.

3. The treatment system of claim 2 wherein at least a portion of the biasing element moves along with the movement of the mammal being treated by the treatment system.

4. The treatment system according to claim 2 wherein the biasing element is connected to the rotating coupling.

5. The treatment system according to claim 2 wherein the biasing element is selected from the group consisting of:
a counterweight system, a spring loaded return coil, a spring, a hydraulic ram and a pneumatic ram.

6. The treatment system according to claim 2 further comprising an arm connected to the rotating coupling and the biasing element supported by the arm.

7. The treatment system according to claim 6 further comprising:
a pivoting connection on the arm wherein the biasing element is connected to the arm to control the movement of the arm about the pivoting connection.

8. The treatment system according to claim 2 wherein the biasing element is configured to maintain a first position when the mammal is receiving therapy in a standing position and a second position when the mammal is receiving therapy in a non-standing position.

9. The treatment system according to claim 1 further comprising:
an upright support connected to the rotating coupling;
another heat exchange bladder for another mammal;
the rotating coupling further comprising:
another rotating side supply conduit, and another rotating side return conduit wherein the another rotating side supply conduit and the another rotating side return conduit are connected to the another heat exchange bladder.

10. The treatment system according to claim 9 wherein the hose connecting the fixed side supply conduit to the heat exchange fluid supply conduit and a hose connecting the fixed side return conduit to the heat exchange fluid return conduit are within or along the upright support.

11. The treatment system according to claim 9 wherein the upright support has a height that places the rotating coupling near the chest level of the mammal receiving therapy from the treatment system.

12. The treatment system according to claim 9 wherein the upright support has a height that places the rotating coupling above the head of the mammal receiving therapy from the treatment system.

13. The treatment system according to claim 9 wherein the upright support and the rotating coupling are positioned to permit the mammal and the another mammal to move in a generally circular path about the upright support.

14. The treatment system according to claim 1 further comprising:
an air bladder on the heat exchange bladder;
an air supply port on the rotating coupling; and
an air supply line between the air bladder and the air supply port.

15. The treatment system according to claim 1 further comprising:
a pharmacological agent in a container in fluid communication with rotating coupling to deliver the pharmacological agent to an outlet on the rotating side of the rotating coupling and a delivery conduit from the outlet on the rotating side to the mammal.

16. The treatment system according to claim 1 further comprising: a container mounted to the rotating side of the rotating coupling, the container including a pharmacological agent and having a delivery conduit in communication with the mammal receiving therapy.

17. The treatment system according to claim 1 further comprising:
an electrical connection providing through the rotating coupling to a component.

18. The treatment system according to claim 17 wherein the electrical connection provides power to the component.

19. The treatment system according to claim 17 wherein the electrical connection provides a signal path for a component.

20. The treatment system according to claim 17 wherein the component is a pump.

21. The treatment system according to claim 17 wherein the component is a sensor on the animal.

22. The treatment system according to claim 21 wherein the sensor provides an indication of the mammal receiving therapy from the therapy system.

23. The treatment system according to claim 21 wherein the sensor is one of an ECG lead, a respiration sensor, a blood pressure sensor or a temperature sensor.

24. The treatment system according to claim 17 wherein the component is a sensor measuring an indication of the therapy system.

25. The treatment system according to claim 1 wherein the hose connecting the heat exchange bladder supply conduit to the rotating side supply conduit or the hose connecting the heat exchange bladder return conduit to the rotating side return conduit has a rotational stiffness of between about 1 in-lb/rad to about 25 in-lb/rad.

26. A treatment system for a mammal, comprising:
a rotating coupling having a fixed side supply conduit, a fixed side return conduit, a rotating side supply conduit, and a rotating side return conduit;
at least one heat exchange bladder for the mammal being treated by the thermal treatment system, the heat exchange bladder having a supply conduit and a return conduit;
a manifold having a main supply conduit, a main return conduit, at least one auxiliary supply conduit and at least one auxiliary return conduit;
a heating or chilling unit having a heat exchange fluid supply conduit and a heat exchange fluid return conduit;
a hose connecting the manifold main supply conduit to the rotating side supply conduit;
a hose connecting the manifold main return conduit to the rotating side return conduit;
a hose connecting the at least one auxiliary supply conduit to the heat exchange bladder supply conduit;
a hose connecting the at least one auxiliary return conduit to the heat exchange bladder return conduit;
a hose connecting the fixed side supply conduit to the heat exchange fluid supply conduit; and
a hose connecting the fixed side return conduit to the heat exchange fluid return conduit.

27. The treatment system for a mammal according to claim 26 wherein the mammal being treated by the system is a horse, further comprising:
a surface on the manifold configured to conform to a portion of the back of the horse and the length of the hose connecting the at least one auxiliary supply conduit to the heat exchange bladder supply conduit and the length of the hose connecting the at least one auxiliary return conduit to the heat exchange bladder return conduit are selected to extend from the manifold to a hoof of the horse.

28. The treatment system for a mammal according to claim 27 wherein in use the hose connecting the manifold main supply conduit to the rotating side supply conduit or the hose connecting the manifold main return conduit to the rotating side return conduit extend from the manifold in a direction towards the rotating coupling.

29. The treatment system for a mammal according to claim 26 wherein the mammal being treated by the system is a horse, further comprising:
a surface on the manifold configured to conform to a portion of the chest of the horse and the length of the hose connecting the at least one auxiliary supply conduit to the heat exchange bladder supply conduit and the length of the hose connecting the at least one auxiliary return conduit to the heat exchange bladder return conduit are selected to extend from the manifold to a hoof of the horse.

30. The treatment system for a mammal according to claim 26 further comprising:
a biasing element connected to the rotating coupling or to at least one of the hose connecting the manifold main supply conduit to the rotating side supply conduit and the hose connecting the manifold main return conduit to the rotating side return conduit.

31. The treatment system of claim 30 wherein the biasing element moves along with the movement of the mammal being treated by the treatment system.

32. The treatment system according to claim 30 wherein the biasing element is connected to the rotating coupling.

33. The treatment system according to claim 30 wherein the biasing element is selected from the group consisting of:
a counterweight system, a spring loaded return coil, a spring, a hydraulic ram and a pneumatic ram.

34. The treatment system according to claim 32 further comprising an arm connected to the rotating coupling and the biasing element supported by the arm.

35. The treatment system according to claim 34 further comprising:
a pivoting connection on the arm wherein the biasing element is connected to the arm to control the movement of the arm about the pivoting connection.

36. The treatment system according to claim 32 wherein the biasing element is configured to maintain a first position when the mammal is receiving therapy in a standing position and a second position when the mammal is receiving therapy in a non-standing position.

37. The treatment system according to claim 26 further comprising:
an upright support connected to the rotating coupling;
another heat exchange bladder for another mammal;
the rotating coupling further comprising:
another rotating side supply conduit, and another rotating side return conduit wherein the another rotating side supply conduit and the another rotating side return conduit are connected to the another heat exchange bladder.

38. The treatment system according to claim 37 wherein the hose connecting the fixed side supply conduit to the heat exchange fluid supply conduit and the hose connecting the fixed side return conduit to the heat exchange fluid return conduit are within or along the upright support.

39. The treatment system according to claim 37 wherein the upright support has a height that places the rotating coupling near the chest level of the mammal receiving therapy from the treatment system.

40. The treatment system according to claim 37 wherein the upright support has a height that places the rotating coupling above the head of the mammal receiving therapy from the treatment system.

41. The treatment system according to claim 37 wherein the upright support and the rotating coupling are positioned to permit the mammal and the another mammal to move in a generally circular path about the upright support.

42. The treatment system for a mammal according to claim 26 wherein the hose connecting the manifold main supply conduit to the rotating side supply conduit or the hose connecting the manifold main return conduit to the rotating side return conduit has a rotational stiffness of between about 1 in-lb/rad to about 25 in-lb/rad.

43. A method of providing therapy to a mammal comprising:
attaching a heat exchange bladder to a portion of the mammal;
placing the heat exchange bladder in fluid communication with a rotating coupling and the rotating coupling in fluid communication with a heating or chilling unit; and
circulating heat exchanging fluid from the heat exchanger through the rotating coupling to the heat exchange bladder while movement performed by the mammal rotates the rotating coupling.

44. The method of providing therapy according to claim 43 further comprising:
displacing the rotating coupling as the mammal moves from a first position to a second position.

45. The method of providing therapy according to claim 44 wherein the first position or the second position the mammal is in a sleeping position.

46. The method of providing therapy according to claim 44 further comprising:
extending a biasing element during the displacing step when the rotating coupling is displaced about a pivot point connected to the rotating coupling.

47. The method of providing therapy according to claim 43 wherein during the circulating step the rotating coupling is maintained above the head of the mammal receiving therapy.

48. The method of providing therapy according to claim 43 wherein during the circulating step the rotating coupling is maintained at about the level of the chest of the mammal receiving therapy.

49. The method of providing therapy according to claim 43 further comprising:
performing the attaching step, the placing step and the circulating step with another mammal such that circulating step is performed using one rotating coupling.

50. The method of providing therapy according to claim 49 further comprising:
moving the mammal and the another mammal in a generally circular path about the rotating coupling during the circulating step.

51. The method of providing therapy according to claim 44 wherein the displacing step further comprises: displacing the rotating coupling about a pivot point.
